(19) United States
(12) Patent Application Publication
(10) Pub. No.: US 2013/0274744 A1
(43) Pub. Date: Oct. 17, 2013

(54) EXTERNAL FIXATION DEVICE FOR FRACTURE TREATMENT OF ANIMALS AND METHOD FOR TREATING FRACTURES OF ANIMALS USING THE DEVICE

(71) Applicant: Tomohiro Ikemizu, Kasai-shi (JP)

(72) Inventor: Tomohiro Ikemizu, Kasai-shi (JP)

(21) Appl. No.: 13/861,061

(22) Filed: Apr. 11, 2013

(30) Foreign Application Priority Data
Apr. 13, 2012 (JP) 2012-091542

(51) Int. Cl. A61B 17/64

(52) U.S. Cl. CPC A61B 17/6441
USPC 606/54

(57) ABSTRACT

An external fixation device 1 for fracture treatment for fixing a fractured affected limb B1 of a quadruped A is configured to include an almost toric ring portion 3 which is to put on periphery C of a shoulder joint and through which the affected limb B1 passes; a supporting portion 4 having right/left walls and a back wall, having upper/lower surfaces and a front face opened, and accompanying the affected limb B1 from a radial head to a distal end of a metacarpal bone; a connecting body 6 for connecting the rear of the ring portion 3 and the back wall of the supporting portion 4; a grounding body 7 being connected to the back wall of the supporting portion 4 and positioned below an opening of the lower surface of the supporting portion; a fixing means 9; and an upper arm supporting section 10.

### BACKGROUND OF THE INVENTION

1. Field of the Invention

The present invention relates to an external fixation device for fracture treatment of animals, in particular, suitable for treatment of distal radioulnar fractures of small quadrupeds such as small dogs, and to a fracture treatment method using the device.

2. Description of the Background Art

Dogs are vulnerable to fractures of distal portions of radius and ulna due to impact of landing (to which they are subjected) when dropping or jumping from a high place. Cats may also suffer from radioulnar fractures due to impact of landing from a high place or of a traffic accident and the like.

As a treatment method of such radioulnar fractures of dogs and cats, a plate internal fixation method, combined use of a plaster cast therapy using an intramedullary nail and a cast, or an external skeletal fixation method and the like are often performed (See Non Patent Document 1, Non Patent Document 2, and Non Patent Document 3, for example.)

It is the plate internal fixation method that has been most widespread among the treatment methods of radioulnar fractures mentioned above. This is because the AO Foundation (“AO” is an acronym standing for Arbeitsgemeinschaft für Osteosynthesefragen, a German word meaning a fundamental clinical research group of fracture treatment) established in 1958 and engaged in research & development, and educational activity on fracture treatment has been disseminating the plate internal fixation method which focuses on recovery of limb function and is based on accurate reduction and compression.

Since the fracture treatment based on the plate internal fixation method is primary healing in which strength of bone adhesion is very weak, treatment takes a long time and usually requires a period of 6 months to 1 year or more. In addition, since the method requires three open surgeries with use of a general anesthetic for mounting a plate, removing partial nail, and removing remaining nails and plate, operative costs are most expensive.

Furthermore, there may occur delayed adhesion or nonunion, or refracture after plate removal (see Non Patent Document 1 and Non Patent Document 2, for example), or plate-induced osteopenia due to hematogenous disorder may be caused even when a leading-edge plate with no compression is used. Thus, external skeletal fixation (see Non-Patent Document 3, for example) and treatment combined with bone regeneration medicine may be provided.

In the case of small dogs, in particular, they have very thin radius and ulna, and as a long abductor muscle of digiti and extensor carpi radialis, for example, change from muscles to tendons toward distal portions of radius and ulna, the amount of muscle in their surrounding region gradually decreases. In the widely used plate internal fixation method, a soft tissue around the radius and ulna is damaged by an open operative procedure in a fracture site or an implant, and hematogenous disorder easily occurs around the fracture site. In addition, since plates suitable for ultra-small dogs having weight of 1 kg or less such as Chihuahuas or Poodles are not commercially available, it is difficult to obtain an appropriately-sized plate.

Furthermore, in the case of ultra-small dogs, since size of radius of a Chihuahua having weight of 600 g, for example, is as small as about a tooth-pick to disposable chopsticks, internal fixation by means of a plate is impossible. Even if external skeletal fixation is adopted in such a case, it is likely that the radius may be broken from a region into which the thinnest pin is inserted or that infection may result from a loosened pin.

In addition, in the external skeletal fixation (see Non-Patent Document 3, for example), while bone adhesion speed is fast and adhesion strength is strong, it requires not only mature, sophisticated techniques and experience in post-operative management, but also more than two surgeries with use of a general anesthetic for insertion and removal of pins.

Furthermore, in the combined use of the plaster cast therapy using an intramedullary nail and a cast, since an intramedullary cavity of a small dog is very narrow, insertion of a pin into marrow may develop hematogenous disorder, resulting in nonunion or even leg amputation in some cases.

Moreover, any surgery requires sophisticated manipulation and costly instruments, and thus demands high costs.

Against the backdrop described above, as an external fixation method effective for treatment of distal radioulnar fractures of small dogs, in particular, in contrast to the Thomas splint consisting of a ring portion to be put on a region surrounding a shoulder joint of an affected limb and a frame portion almost shaped like a letter U and including front and rear rods drooping from the ring portion, there are a method using an external fixation device for fracture treatment (hereinafter referred to as a type-A external fixation device) in which a plate-like splint made of aluminum alloy is diagonally attached to the frame portion (see Non-Patent Document 4, Non Patent Document 5, Non Patent Document 6, and Non Patent Document 7, for example) or a method using an external fixation device for fracture treatment (hereinafter referred to as type-B external fixation device) which has been modified to fix a gutter-like splint made of synthetic resin to the plate-like splint so as to support the affected limb (see Non-Patent Document 8 and Non Patent Document 9, for example). With them, radioulnar fractures of small dogs can be treated in a relatively safe and reliable manner by externally fixing an affected limb using the external fixation device for fracture treatment following reduction of the fracture site through non-invasive reduction.

### CITATION LIST

### Non Patent Literature

[Non Patent Document 1] STEPHAN M. PERREN, “Fracture Healing the Evolution of Our Understanding” Acta Chir Orthop Traumatol Cech. 75(4), p 241-246, 2008
[Non Patent Document 2] Harada, Yuguchi, Itoh, Sasaki, Abe, Izumisawa, “Study of Plate Fixation Method to Radioulnar Fracture of Dog”, The Journal of the Hokkaido Veterinary Medical Association, Vol. 52, No. 8, August 2008, P 79
[Non Patent Document 3] Johnson A L & Egger E L, “Biomechanics and biology of fracture healing with external skeletal fixation”, Comp Cont Educ Pract Vet 20, p 487-502, 1998
[Non-Patent Document 4] Katoh, Ikemizu, Muroya, “One case in which a radioulnar fracture of Pomeranians is healed through application of modified procedure of the Thomas splint fixation”, The Journal of the Hokkaido Veterinary Medical Association, Vol. 52, No. 8, August 2008, P 79
[Non Patent Document 5] Ikemizu, Katoh, Muroya, “Experience in Using modified procedure of the Thomas splint fixation in eight fracture cases of dogs and cats”, The Journal of the Hokkaido Veterinary Medical Association, Vol. 52, No. 8, August 2008, P 80
[Non Patent Document 6] Shirahama, Ikemizu, Katoh, Muroya, Yoshioka, “Two cases in which radioulnar fractures of Toy poodles were healed by applying modified procedure of the Schroeder-Thomas splint fixation (i-T method)”, The Journal of the Hokkaido Veterinary Medical Association, Vol. 53, No. 8, August 2009, P 76
[Non Patent Document 7] Ikemizu, Katoh, Muroya, Shirahama, Yoshioka, “One case in which the bilateral radioulnar fractures of a smooth Chihuahua was healed through combined use of mini exposure technique and modified procedure of the Schroeder-Thomas splint fixation (i-T method)”, The Journal of the Hokkaido Veterinary Medical Association, Vol. 53, No. 8, August 2009, P 76
[Non-Patent Document 8] Yasuda, Ikemizu, Katoh, Yoshioka, Shirahama, Muroya, Kitamori “Healing performance of distal radioulnar fractures of small dogs through the external fixation method using the Schroeder-Thomas Splint and Ikemizu-Thomas Splint”, The Journal of the Hokkaido Veterinary Medical Association, Vol. 54, No. 8, August 2010, P 94
[Non Patent Document 9] Ikemizu, Yasuda, Katoh, Kitamori, “Healing performance of distal radioulnar fractures of small dogs through resilient fixation method using the Ikemizu-Thomas Splint”, The Journal of the Hokkaido Veterinary Medical Association, Vol. 54, No. 8, August 2010, P 94

### SUMMARY OF THE INVENTION

While the type-A external fixation device and the type-B external fixation device have the characteristics described above, they have problems that fixation of a ring portion to be put on a region surrounding a shoulder joint of an affected limb easily loosens; it is difficult to fix an elbow joint and periphery of an upper arm; a plate-like splint made of aluminum alloy and a gutter-like splint made of synthetic resin easily come off due to external impact; a front frame (a rod on the front side) is obstructive when the radius and ulna are fixed in the gutter-like sprint; combined use with the external skeletal fixation method is difficult; weight bearing is not possible, and the like. Therefore, there is still a need to modify those external fixation devices.

What the present invention intends to solve in light of the circumstances described above lies in provision of an external fixation device for fracture treatment of animals which can be easily attached to an affected limb, fixed in a stable and reliable manner, used combined with the external skeletal fixation method, and increase callus to shorten a period of treatment by providing a fracture site with stimulation due to weight bearing when some callus has been formed during the course of treatment, and of a method for treating fractures of animals using the device.

Taking advantage of his experience in treatment of distal radioulnar fractures of small dogs by means of the type-A and type-B external fixation devices, the inventor of this application could come to completion of the present invention through study and embodiment of a structure of an external fixation device which is most suitable for a flexible fixation method without accurate reduction used as a secondary healing method with callus having strong adhesion strength, to modify the devices by prototyping and to evaluate application of the device to treatments.

Specifically, an external fixation device for fracture treatment of animals according to the present invention (external fixation device for fracture treatment of animals of the invention 1) is an external fixation device for fracture treatment used in treatment of forelimb fractures of quadrupeds for fixing an affected limb in order to solve the problems described above, the device including an almost toric ring portion which is to put on periphery of a shoulder joint and through which the affected limb passes; a supporting portion having right/left walls and a back wall, having upper/lower surfaces and a front face opened, and accompanying the affected limb from a radial head to at least a distal end of the radius, and supporting the affected limb so that flexible micromotion of fracture site of the affected limb can be produced with it loosely placed in the supporting portion; a connecting body for connecting the rear of the ring portion and the back wall of the supporting portion; and a grounding body being connected to the back wall of the supporting portion and positioned below the opening of the lower surface of the supporting portion, and having traction means for linear traction of the affected limb downward straight while keeping the entire affected limb stretched.

With such a configuration, since with a fracture site of the affected limb loosely put in the supporting portion having right/left walls and a back wall and having upper/lower surfaces and a front face opened, the affected limb is supported by the supporting portion accompanying the affected limb from the radial head to at least the distal end of the radius, not only the part below the radial head is stably fixed, but also the fracture site finely and flexibly vibrates and the blood circulation is preserved without being compressed, which thereby can facilitate formation of callus. Thus, this device is suitable for fracture treatment wherein the device is used for a certain period of time to an extent that bone density is not reduced, and thereby facilitates formation of callus.

In addition, since the grounding body includes the traction means for linear traction of the affected limb downward straight while keeping the entire affected limb stretched, and the affected limb is stably and reliably pulled to right below, it is possible to maintain the condition in which an edge of fracture is non-precisely reduced although it is reduced.

Furthermore, since not only the connecting body connects the rear of the ring portion and the back wall of the supporting portion so that they are firmly integrated, but also the front face of the supporting portion is an opening with no obstruction such as a frame body in front, a veterinarian can easily perform a physical examination, procedure, an X-ray examination and the like, and combined use with the external skeletal fixation method is also allowed.

In addition, an external fixation device for fracture treatment of animals according to the present invention (external fixation device for fracture treatment of animals of the invention 2) is an external fixation device for fracture treatment used in treatment of forelimb fractures of quadrupeds for fixing a fractured affected limb in order to solve the problems described above, the device including an almost toric ring portion which is to put on periphery of a shoulder joint and through which the affected limb passes; a supporting portion having right/left walls and a back wall, having upper/lower surfaces and a front face opened, and accompanying the affected limb from radial head to at least a distal end of the radius, and supporting the affected limb so that flexible micromotion of fracture site of the affected limb can be produced with it loosely placed in the supporting portion; a connecting body connecting the rear of the ring portion and the back wall of the supporting portion; and a metacarpal pad supporting plate being connected to the back wall of the supporting portion, and tilting forward as it accompanies a metacarpal pad of the affected limb downward from the lower end of the supporting portion.

With such a configuration, since with a fracture site of the affected limb loosely put in the supporting portion having right/left walls and a back wall and having upper/lower surfaces and a front face opened, the affected limb is supported by the supporting portion accompanying the affected limb from the radial head to at least the distal end of the radius, not only the part below the radial head is stably fixed, but also the fracture site finely vibrates and the blood circulation is preserved without being compressed, which thereby can facilitate formation of callus, and digital pads ground with the metacarpal pad of the affected limb being supported by the metacarpal pad supporting plate. Thus, the device is suitable for fracture treatment in which relatively small weight load is applied and stimulation caused by fine vibration resulting from the weight bearing is provided to the fracture site, which thereby contributes to maturing and increasing of the callus.

In addition, since not only the connecting body connects the rear of the ring portion and the back wall of the supporting portion so that they are firmly integrated, but also the front face of the supporting portion is an opening with no obstruction such as a frame body in front, a veterinarian can easily perform a physical examination, procedure, an X-ray examination and the like, and combined use with the external skeletal fixation method is also allowed.

In addition, an external fixation device for fracture treatment of animals according to the present invention (external fixation device for fracture treatment of animals of the invention 3) is an external fixation device for fracture treatment used in treatment of forelimb fractures of quadrupeds for fixing a fractured affected limb in order to solve the problems described above, the device including an almost toric ring portion which is to put on periphery of a shoulder joint and through which the affected limb passes; a supporting portion having right/left walls and a back wall, having upper/lower surfaces and a front face opened, and accompanying the affected limb the radius from radial head to at least a distal end of the radius, and supporting the affected limb so that flexible micromotion of fracture site of the affected limb can be produced with it loosely placed in the supporting portion; and a connecting body for connecting the rear of the ring portion and the back wall of the supporting portion.

With such a configuration, since with a fracture site of the affected limb loosely put in the supporting portion having right/left walls and a back wall and having upper/lower surfaces and a front face opened, the affected limb is supported by the supporting portion accompanying the affected limb from the radial head to at least the distal end of the radius, not only the part below the radial head is stably fixed, but also the fracture site finely vibrates and the blood circulation is preserved without being compressed, which thereby can facilitate formation of callus, and a metacarpal pad as well as digital pads of the affected limb ground. Thus, the device is suitable for fracture treatment in which since, relatively large weight load is applied, and stimulation caused by fine vibration resulting from the weight bearing is provided to the fracture site, which thereby contributes to maturing and increasing the callus.

In addition, since not only the connecting body connects the rear of the ring portion and the back wall of the supporting portion so that they are firmly integrated, but also the front face of the supporting portion is an opening with no obstruction such as a frame body in front, a veterinarian can easily perform a physical examination, procedure, an X-ray examination and the like, and combined use with the external skeletal fixation method is also allowed.

It is preferable that the device includes fixation means to connect and fix the ring portion to a forelimb opposite to the affected limb, with the ring portion put on the periphery of the shoulder joint of the affected limb.

With such a configuration, since the ring portion put on the periphery of the shoulder joint of the affected limb is connected and fixed to the forelimb opposite to the affected limb by fixation means, the fixed ring portion can be prevented from becoming loose even if an animal loses weight during fracture treatment.

In addition, it is also preferable that the device include an upper arm supporting section which droops from the front of the ring portion to an elbow joint of the affected limb and front side of the upper arm.

With such a configuration, the upper arm supporting section drooping to the elbow joint of the affected limb and the front side of the upper arm is used, and the elbow joint (point of the elbow) of the affected limb and the upper arm part can be fixed to the upper arm supporting section by fabric adhesive tapes. Thus, the affected limb can be stably supported because the affected limb can be easily pulled down straight with the elbow joint and the upper arm periphery easily fixed.

Furthermore, it is preferable that the supporting portion is made of transparent synthetic resin.

With such a configuration, inflammation, swelling, congestion, and the like of the skin of the affected limb can be easily checked during fracture treatment.

A method for treating fractures of animals according to the present invention is a method for treating forelimb fractures of quadrupeds in order to solve the problems described above, the method including: a reduction step of reducing an affected limb by linear traction or toggling an edge of the fracture; a callus formation facilitation step of facilitating formation of callus to an extent that bone density is not reduced, with the use of the external fixation device for fracture treatment of animals of the invention 1, by linear traction of the affected limb with the traction means and grounding the grounding body; a callus maturing and increasing step based on weight bearing of maturing and increasing callus, with the use of the external fixation device for fracture treatment of animals of the invention 2, by grounding digital pads of the affected limb to provide the fracture site with stimulation resulting from the weight bearing; and a callus maturing and increasing step based on weight bearing of maturing and increasing callus, with the use of the external fixation device for fracture treatment of animals of the invention 3, by grounding a metacarpal pad and the digital pads of the affected limb to provide the fracture site with stimulation resulting from the weight bearing.

With such a configuration, after the callus formation is facilitated by using the external fixation device for fracture treatment of animals of the invention 1 to the extent that bone density is not reduced, the external fixation device for fracture treatment of animals of the invention 1 is removed and replaced with the external fixation device for fracture treatment of animals of the invention 2, thereby grounding the digital pads with the metacarpal pad of the affected limb being supported by the metacarpal pad supporting plate to mature and increase the callus while providing stimulation caused by fine vibration resulting from relatively small weight bearing. Effective fracture treatment is performed at this stage, so that maturing and increasing of the callus advance to a state in which a certain amount of the callus is formed. Then, the external fixation device for fracture treatment of animals of the invention 2 is removed and replaced with the external fixation device for fracture treatment of animals of the invention 3, thereby grounding the metacarpal pad and the digital pads of the affected limb, and being able to perform effective fracture treatment to mature and increase the callus while providing stimulation caused by fine vibration resulting from relatively large weight bearing.

The fracture treatment method to be performed by using in a phased manner the external fixation device for fracture treatment of animals of the invention 1, the external fixation device for fracture treatment of animals of the invention 2, and the external fixation device for fracture treatment of animals of the invention 3 not only allows safe and early healing involving full recovery of functions but also enables the risk of refracture to be minimized.

In addition, in the external fixation devices for fracture treatment of animals of the invention to be used in the method for treating fractures of animals, the connecting body connects the rear of the ring portion and the back wall of the supporting portion so that they are firmly integrated, and the front face of the supporting portion is an opening with no obstruction such as a frame body in front. Thus, a veterinarian can easily perform a physical examination, procedure, an X-ray examination and the like, and combined use with the external skeletal fixation method is also allowed.

In addition, a method for treating fractures of animals according to the present invention is a method for treating forelimb fractures of quadrupeds in order to solve the problems described above, the method including: a reduction step of reducing an affected limb by linear traction or toggling an edge of the fracture; a callus formation facilitation step of facilitating formation of callus to an extent that bone density is not reduced, with the use of the external fixation device for fracture treatment of animals of the invention 1, by linear traction of the affected limb with the traction means and grounding the grounding body; and a callus maturing and increasing step based on weight bearing of maturing and increasing callus, with the use of the external fixation device for fracture treatment of animals of the invention 3, by grounding a metacarpal pad and digital pads of the affected limb to provide the fracture site with stimulation resulting from the weight bearing.

With such a configuration, if formation of callas has been facilitated using the external fixation device for fracture treatment of animals of the invention 1 and a certain amount of callus has already been formed, specifically, for example, if formation of callus is fast in the case of a fracture of a young animal, the external fixation device for fracture treatment of animals of the invention 1 is removed and replaced with the external fixation device for fracture treatment of animals of the invention 3 to mature and increase the callus and to be able to provide effective fracture treatment while grounding a metacarpal pad and digital pads of the affected limb and providing stimulation caused by fine vibration resulting from relatively large weight bearing.

The fracture treatment method to be performed by using in a phased manner the external fixation device for fracture treatment of animals of the invention 1 and the external fixation device for fracture treatment of animals of the invention 3 not only allows safe and early healing involving full recovery of functions but also enables the risk of refracture to be minimized.

In addition, in the external fixation devices for fracture treatment of animals of the invention to be used in the method for treating fractures of animals, the connecting body connects the rear of the ring portion and the back wall of the supporting portion so that they are firmly integrated, and the front face of the supporting portion is an opening with no obstruction such as a frame body in front. Thus, a veterinarian can easily perform a physical examination, procedure, an X-ray examination and the like, and combined use with the external skeletal fixation method is also allowed.

It is also preferable to include fixing means to connect and fix the ring portion to a forelimb opposite to the affected limb, with the ring portion of the external fixation device for fracture treatment put on the periphery of the shoulder joint of the affected limb.

With such a configuration, since the ring portion put on the periphery of the shoulder joint of the affected limb connected and fixed to the forelimb opposite to the affected limb, appropriate fracture treatment can be performed while preventing the fixed ring portion from becoming loose even if an animal loses weight during fracture treatment.

In addition, in the method for treating fractures of animals, it is preferable to include an upper arm supporting section drooping from the front of the ring portion of the external fixation device for fracture treatment to an elbow joint of the affected limb and the front side of the upper arm.

With such a configuration, the upper arm supporting section drooping to an elbow joint of the affected limb and the front side of the upper arm is used, and the elbow joint (point of the elbow) of the affected limb and the upper arm part can be fixed to the upper arm supporting section by fabric adhesive tapes. Thus, appropriate fracture treatment can be performed while maintaining a state in which the affected limb is stably supported, because the affected limb can be easily pulled to the right below with the elbow joint and the upper arm periphery easily fixed.

Furthermore, in the method for treating fractures of animals, it is preferable that the supporting portion of the external fixation device for fracture treatment is made of transparent synthetic resin.

With such a configuration, inflammation, swelling, congestion, and the like of the skin of the affected limb can be easily checked during fracture treatment.

As described above, according to the external fixation device for fracture treatment of animals and the method for treating fractures of animals using the device according to the present invention, since with a fracture site of the affected limb loosely in the supporting portion of the external fixation device for fracture treatment of animals, the affected limb is supported by the supporting portion accompanying the affected limb from the radial head to at least the distal end of the radius, not only the part below the radial head is stably fixed, but also the blood circulation is preserved without being compressed, which thereby can facilitate formation of callus. Thus, safe and early healing involving full recovery of functions can be implemented in treatment of distal radioulnar fractures of quadrupeds by using in a phased manner the external fixation device for fracture treatment of animals of the invention 1, the external fixation device for fracture treatment of animals of the invention 2, and the external fixation device for fracture treatment of animals of the invention 3, or by using in a phased manner the external fixation device for fracture treatment of animals of the invention 1 and the external fixation device for fracture treatment of animals of the invention 3. In addition, the risk of refracture can also be minimized, and remarkable effects can be achieved that the device can be easily mounted to the affected limb, and the device can be stably and reliably fixed and used with the external skeletal fixation method.

### BRIEF DESCRIPTION OF THE DRAWINGS

FIG. 1 is a view showing a dog, when viewed from the front, which has an external fixation device for fracture treatment of animals according to an embodiment 1 of the present invention attached to its left forelimb.

FIG. 2 is a view showing the dog when viewed from the left side.

FIG. 3 is a perspective view of the external fixation device for fracture treatment of animals according to the embodiment 1 of the present invention.

FIG. 4 is a longitudinal sectional view of the device when viewed from the left side.

FIG. 5(a) is a view of the device when viewed from the rear. FIG. 5(b) is a sectional view of FIG. 5(a) taken along line X1-X1.

FIG. 6 is an exploded perspective view of the device.

FIGS. 7(a) to 7(d) are diagrammatic views, viewed from the left side, showing one example of a method for applying fabric adhesive tapes to an affected limb when the external fixation device for fracture treatment of animals according to the embodiment 1 of the present invention is used. FIG. 7(a) shows a state in which front and back two fabric adhesive tapes are applied in an overlapping manner to a distal portion of an edge of fracture, and front and back two fabric adhesive tapes are applied in an overlapping manner to a proximal portion of the edge of fracture. FIG. 7(b) and FIG. 7(c) show a state in which the distal and proximal edges of fracture are reinforced by horizontally wound fabric adhesive tapes. FIG. 7(d) shows an example of fabric adhesive tapes to be applied to fix a supporting portion of an external fixation device for fracture treatment.

FIG. 8 is a view showing a dog, when viewed from the front, which has an external fixation device for fracture treatment of animals according to an embodiment 2 of the present invention attached to its left forelimb.

FIG. 9 is a view showing the dog when viewed from the left side.

FIG. 10 is a perspective view of the external fixation device for fracture treatment of animals according to the embodiment 2 of the present invention.

FIG. 11 is a longitudinal sectional view of the device when viewed from the left side.

FIG. 12(a) is a view of the device when viewed from the rear. FIG. 12(b) is a sectional view of FIG. 12(a) taken along line X2-X2.

FIG. 13 is an exploded perspective view of the device.

FIGS. 14(a) to 14(c) are diagrammatic views, when viewed from the left side, showing one example of a method for cutting fabric adhesive tapes and applying new fabric adhesive tapes when the external fixation device for fracture treatment of animals of the embodiment 1 of the present invention is removed and replaced with the external fixation device for fracture treatment of animals of the embodiment 2 or embodiment 3. FIG. 14(a) shows a location where the fabric adhesive tape is cut. FIG. 14(b) shows a state after the fabric adhesive tape is cut. FIG. 14(c) shows an example of the fabric adhesive tape to be applied to fix to the supporting portion of the external fixation device for fracture treatment of animals of the embodiment 2 or 3 of the present invention.

FIG. 15 is a view showing a dog, when viewed from the front, which has an external fixation device for fracture treatment of animals according to the embodiment 3 of the present invention attached to its left forelimb.

FIG. 16 is a view showing the dog when viewed from the left side.

FIGS. 17(a) to 17(c) are enlarged views of a main section, when viewed from the left side, of a grounded state of an affected limb of the dog, which has the external fixation device for fracture treatment of animals according to the embodiments 2 and 3 of the present invention attached. FIG. 17(a) shows a case in which the dog has the external fixation device for fracture treatment of animals according to the embodiment 2 of the present invention. FIG. 17(b) shows a case in which the dog has the external fixation device for fracture treatment of animals according to the embodiment 3 of the present invention. FIG. 17(c) shows a case in which a metacarpal pad supporting plate of the external fixation device for fracture treatment of animals of the embodiment 2 of the present invention is bent backward to serve as the external fixation device for fracture treatment of animals of the embodiment 3 of the present invention.

FIGS. 18(a) to 18(d) are schematic views of anterior-posterior X-ray images showing a healing process of a transverse fracture of left radius and ulna due to flexural load at a site of approximately 20% from a distal end of the radius of the toy dog type. FIG. 18(a) shows the X-ray image when the left radius and ulna are fractured. FIG. 18(b) shows the X-ray image when an edge of fracture is reduced. FIG. 18(c) shows the X-ray image when callus is formed. FIG. 18(d) shows the X-ray image of a bone remodeling period.

FIGS. 19(a) to 19(d) are schematic views of anterior-posterior X-ray images showing a healing process of an oblique fracture of right radius and ulna due to compressive load at a site of approximately 20% from a distal end of the radius of the toy dog type. FIG. 19(a) shows the X-ray image when the right radius and ulna are fractured. FIG. 19(b) shows the X-ray image when an edge of fracture is reduced. FIG. 19(c) shows the X-ray image when callus is formed. FIG. 19(d) shows the X-ray image of a bone remodeling period.

FIGS. 20(a) to 20(d) are schematic views of X-ray images in the right-left direction (internal-external direction) showing a healing process of a transverse fracture of left radius and ulna due to flexural load at a site of approximately 18% from a distal end of the radius of a Toy Poodle. FIG. 20(a) shows the X-ray image when the left radius and ulna are fractured. FIG. 20(b), FIG. 20(c), and FIG. 20(d) show the X-ray images when callus is formed. FIG. 20(e) shows the X-ray image of a bone remodeling period.

FIGS. 21(a) to 21(d) are schematic views of anterior-posterior X-ray images showing a healing process of a transverse fracture of left radius and ulna due to flexural load at a site of approximately 18% from a distal end of the radius of a Toy Poodle. FIG. 21(a) shows the X-ray image when the left radius and ulna are fractured. FIG. 21(b) shows the X-ray image when an edge of fracture is reduced. FIG. 21(c), FIG. 21(d) and FIG. 21(e) shows the X-ray images when callus is formed. FIG. 21(f) shows the X-ray image of a bone remodeling period.

FIGS. 22(a) to 22(f) are schematic views of X-ray images in the right-left direction (internal-external direction) showing a healing process of an oblique fracture of right radius and ulna due to compressive load at a site of approximately 10% from a distal end of the radius of a Toy Poodle. FIG. 22(a) shows the X-ray image when the right radius and ulna are fractured. FIG. 22(b) shows the X-ray image when an edge of fracture is reduced. FIG. 22(c), FIG. 22(d), and FIG. 22(e) show the X-ray images when callus is formed. FIG. 22(f) shows a the X-ray image of bone remodeling period.

FIGS. 23(a) to 23(f) are schematic views of anterior-posterior X-ray images showing a healing process of an oblique fracture of right radius and ulna due to compressive load at a site of approximately 10% from a distal end of the radius of a Toy Poodle. FIG. 23(a) shows the X-ray image when the right radius and ulna are fractured. FIG. 23(b) shows the X-ray image when an edge of fracture is reduced. FIG. 23(c), FIG. 23(d), and FIG. 23(e) show the X-ray images when callus is formed. FIG. 23(f) shows the X-ray image of a bone remodeling period.

FIGS. 24(a) to 24(e) are schematic views of anterior-posterior X-ray images showing a healing process of an oblique fracture of right radius and ulna due to compressive load at a site of approximately 17% from a distal end of the radius of a Chihuahua. FIG. 24(a) shows the X-ray image when union of the right radius and ulna is faulty. FIG. 24(b), FIG. 24(c), and FIG. 24(d) show the X-ray images when callus is formed. FIG. 24(e) shows the X-ray image of a bone remodeling period.

FIGS. 25(a) to 25(c) are schematic views of anterior-posterior X-ray images of an oblique fracture of right radius and ulna due to compressive load at a site of approximately 17% from a distal end of the radius of a Chihuahua. FIG. 25(a) shows the X-ray image when the right radius and ulna are fractured. FIG. 25(b) and FIG. 25(c) show the X-ray images when union of the right radius and ulna is faulty.

### DESCRIPTION OF THE PREFERRED EMBODIMENTS

When radial and elbow bones are initially fractured, there exists a blood tumor formed by bleeding from a fracture site, in a gap between a proximal bone section and a dismal bone section of the fractured radius and ulna immediately after the fracture. The blood tumor secretes cytokine, a substitute which triggers starting of healing of the fracture and receives supply of newborn blood vessels from the artery around the radius and ulna to serve as a footing of callus. If an incisional surgery is performed and the blood tumor is removed, the fracture healing does not start, and no callus is formed.

The present invention aims to enable healing in a relatively short period of time such as from 4 weeks to about 3 months at most, ensure safe healing involving full recovery of functions, and minimize risk of refracture, by providing an external fixation device for fracture treatment of animals most suitable for flexible fixation of non-precisely reduced fractures, and which is used as a secondary healing method with callus having strong adhesion strength.

Note that the following description is provided with reference to a dog shown as an example of quadrupeds, and that an external fixation device for fracture treatment of animals is based on a state in which it is attached to a left forelimb of the dog. A direction from the tail side to the head side of the dog is referred to as front, an opposite direction thereto is referred to as back, and the right and left directions refer to those facing the front.

### Embodiment 1

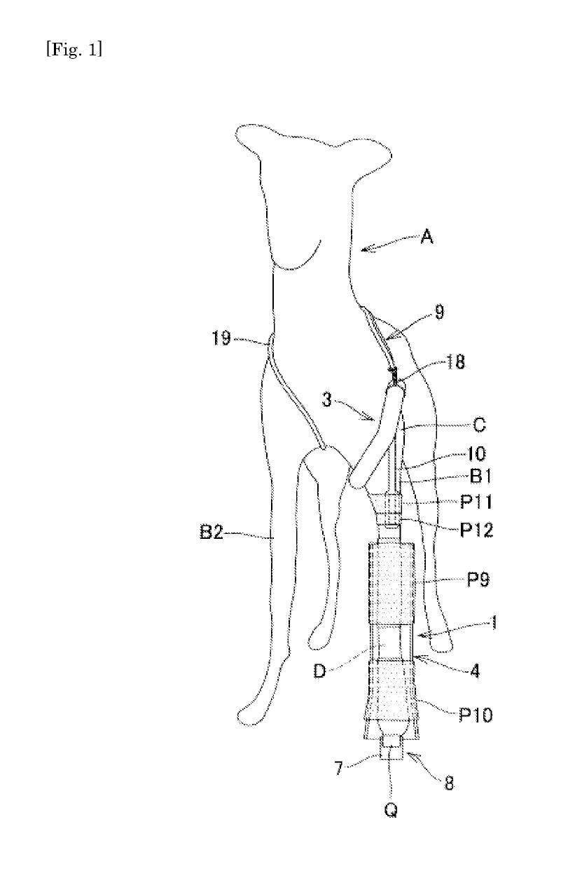

As shown in FIG. 1 to FIG. 6, an external fixation device 1 for fracture treatment of animals according to an embodiment 1 of the present invention is attached to an affected limb B1, for example, a left forelimb, suffering from a distal radioulnar fracture, of a dog A, and flexibly fixes the affected limb B1, the device including an almost toric ring portion 3 which is to put on periphery C of a shoulder joint and through which the affected limb B1 passes; a supporting portion 4 positioned below the ring portion 3, having right/left walls 14A, 14B and a back wall 14C, having upper/lower surfaces and a front face opened, and accompanying the affected limb B1 from radial head to a distal end of metacarpal bone, and supporting the affected limb B1 so that flexible micromotion of fracture site of the affected limb B1 can be produced with it loosely placed in the supporting portion 4; a connecting body 6 for connecting the rear of the ring portion 3 and the back wall 14C of the supporting portion 4; and a grounding body 7 being connected to the back wall 14C of the supporting portion 4 and positioned below the opening of the lower surface of the supporting portion 4, and including traction means 8 for linear traction of the affected limb B1 downward straight while keeping the entire affected limb B1 stretched. Note that the supporting portion 4 has only to accompany the affected limb B1 from the radial head to at least the distal end of radius. If an upper end of the supporting portion 4 is located above the radial head, a point of the elbow rubs against an inner surface of the back wall 14C of the supporting portion 4. If a lower end of the supporting portion 4 is located above the distal end of radius, fixing of a region below the radial head easily becomes unstable.

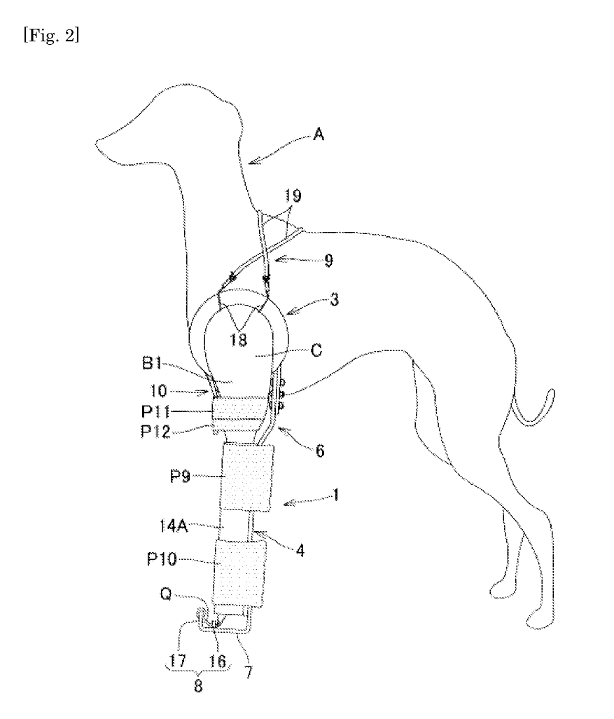

Next, with reference to FIG. 3 to FIG. 6, a structure of the external fixation device 1 for fracture treatment will be described in detail.

The ring portion 3 consists of circular wire rods, such as aluminum alloy wire, stainless steel wire, or titanium alloy wire and the like, around which cotton gauzes are wound and then bandage is further wound, or the circular wire rods whose periphery is covered by a cushioning material such as sponges and the like. Specifically, the ring portion 3 is an almost toric cushioned member having thickness, and, as shown in FIG. 5, an almost lower half of the ring portion 3 is flexed about 20° to the right side (chest side of the dog A). Note that the flexing angle typically ranges from about 10 to 30° since the ring portion 3 is flexed to suit a dog to which the external fixation device 1 for fracture treatment is attached.

Then, to the front and back of the ring portion 3 are mounted an upper arm supporting section 10 made of aluminum alloy and a mounting plate 12, the upper arm supporting section 10 drooping from the front of the ring portion 3, and the mounting plate 12 drooping from the rear of the ring portion 3.

In addition, to the front and rear of the upper part of the ring portion 3 are attached annular strings 18, 18. One end of a fixing string 19 is locked to the annular string 18. The annular strings 18, 18 and the fixing strings 19, 19 constitute fixation means 9 to be described below.

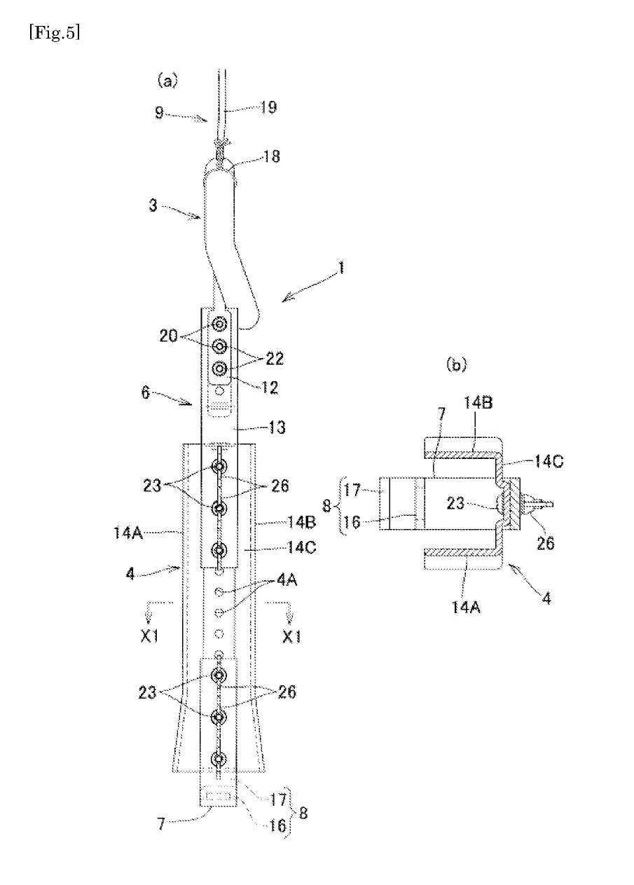

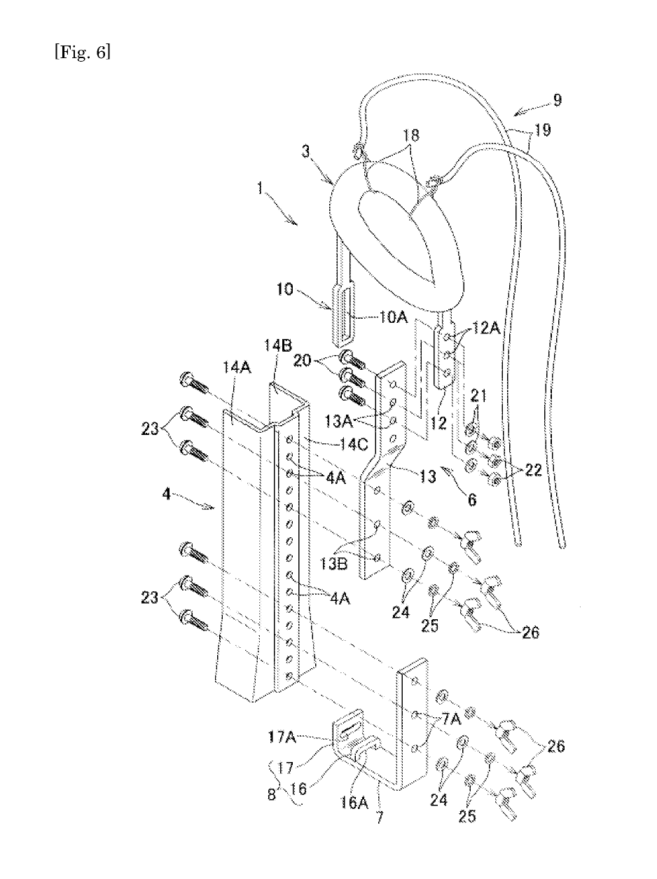

The supporting portion 4 has a cross section almost shaped like a staple with the front side being open and consisting of a left wall 14A, a right wall 14B, and a back wall 14C. The supporting portion 4 is formed to be long to accompany the affected limb B1 from a radial head to a distal end of metacarpal bone. The supporting portion 4 widens in the right-left direction toward the lower ends of the right/left walls 14A, 14B so that the supporting portion 4 does not interfere with the metacarpal bone and the like while accompanying the affected limb B1. As shown in FIG. 6, front-back through-holes 4A, 4A, . . . vertically line up at the center of the right-left direction of the back wall 14C.

In addition, the supporting portion 4 is made of synthetic resin. In particular, the supporting portion 4 made of transparent synthetic resin, such as vinyl chloride resin, polycarbonate or acrylic resin, enables inflammation, swelling, congestion, and the like of the affected limb to be checked. Therefore, it is more preferable embodiment to make the supporting portion of transparent synthetic resin.

As shown in FIG. 6, front-back through-holes 12A, 12A . . . vertically line up on the mounting plate 12 drooping from the rear of the ring portion 3.

In addition, a connecting plate 13 is a plate made of aluminum alloy, having an uneven form in which the upper part is relatively displaced backward and the lower part forward. Front-back through-holes 13A, 13A, . . . line up on the upper part, while the front-back through-holes 13B, 13B, . . . vertically line up on the lower part.

Thus, ring portion 3 and the supporting portion 4 are connected and fixed so that they are firmly integrated, by inserting set screws 20 from the front through the through-holes 13A, the through-holes 12A, and plain washers 21 to threadably mount the set screws 20 to nuts 22, with the upper rear face of the connecting plate 13 abutting against the front face of the mounting plate 12, and by inserting set screws 23 from the front through through-holes 4A, the through-holes 13B, plain washers 24, and spring washers 25 to threadably mount the set screws 23 to butterfly nuts 26, with the front face of the connecting plate 13 abutting against the upper rear face of the back wall 14C.

Here, the mounting plate 12 and the connecting plate 13, and the set screws 20, the plain washers 21 and the nuts 22, the set screws 23, the plain washers 24, the spring washers 25, and the butterfly nuts 26 constitute the connecting body 6 connecting the rear of the ring portion 3 and the back wall 14C of the supporting portion 4.

Note that as shown in FIG. 5(b) and FIG. 6, since the center part in the right-left direction of the back wall 14C of the supporting portion 4 is made a backward lower stage, and the vertically long lower stage accommodates heads of the set screws 23, 23, . . . , the heads of the set screws 23, 23, . . . do not press the affected limb B1 when it is housed in the supporting portion 4.

The grounding body 7 is an aluminum alloy plate, for example, and almost shaped like the letter J when viewed from the left side. Since front-back through-holes 7A, 7A, . . . vertically line up on the vertically extending plate section, the supporting portion 4 and the grounding body 7 are connected and fixed so that they are firmly integrated, by inserting the set screws 23 from the front through the through-holes 4A, through-holes 7A, the plain washers 24, and the spring washers 25 to threadably mount the set screws 23 to the butterfly nuts 26, with the front face of the vertically-extending plate section of the grounding body 7 abutting against the lower rear face of the back wall 14C of the supporting portion 4.

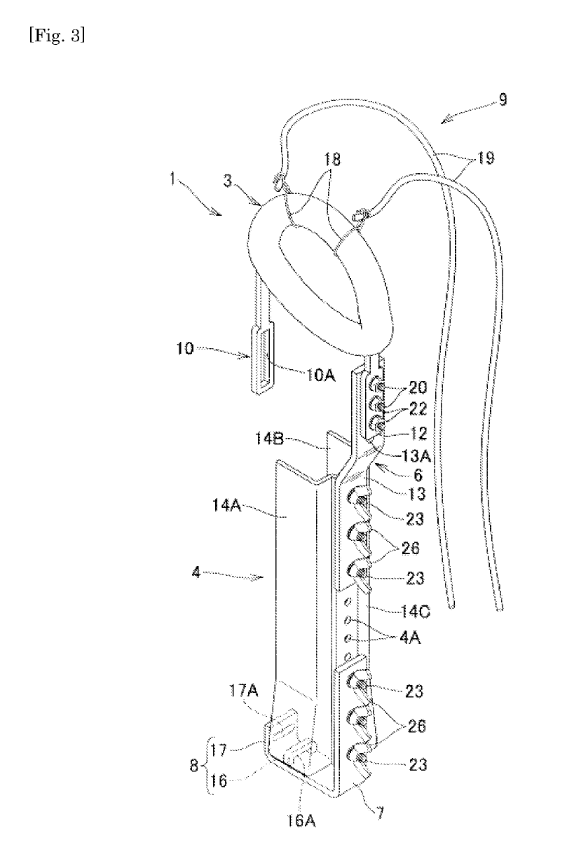

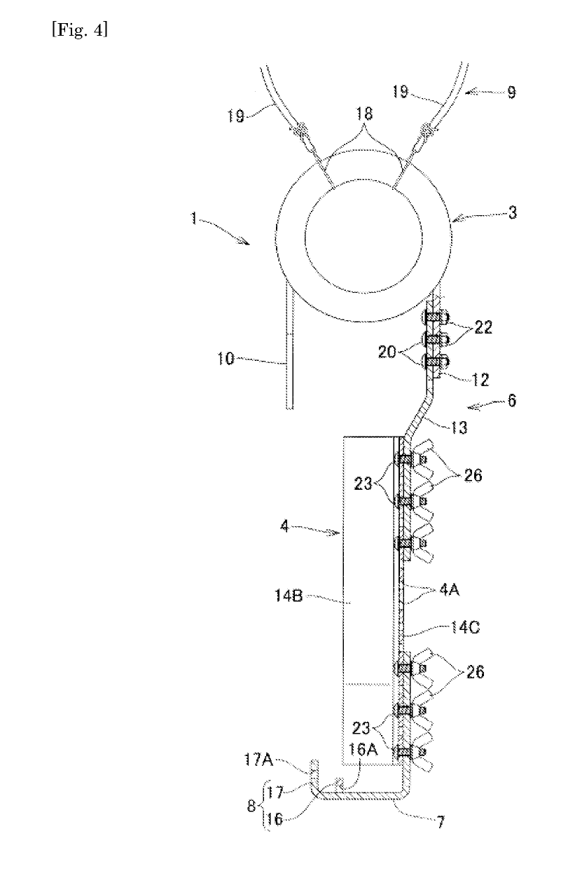

In addition, at a front-side position (see FIG. 4 and FIG. 5(b)) underneath a line connecting the right/left walls 14A, 14B of the supporting portion 4 on the top face of the front-back extending plate section of the grounding body 7 almost shaped like the letter J, a hook section 16 in which a slot 16A being long in the right-left direction is formed in the front-back direction is provided. The front end of the grounding body 7 is bent upward to form a locking section 17. A slot 17A being long in the right-left direction is formed at the vertically central position of the locking section 17. The hook section 16 and the locking section 17 constitute traction means 8 for linear traction of the affected limb B1 as described later.

Four through-holes 13A, 13A, . . . are formed on the upper part of the connecting plate 13 and a number of (15 in the embodiment) through-holes 4A, 4A, . . . are also formed on the back wall 14C of the supporting portion 4. Making a selection among them, and connecting and fixing the connecting plate 13 and the mounting plate 12, the connecting plate 13 and the supporting portion 4, and the supporting portion 4 and the grounding body 7 enable delicate adjustment of length of the external fixation device 1 for fracture treatment in accordance with length of the associated limb B1. In particular, a connecting position for connecting the connecting plate 13 and the grounding body 7 to the supporting portion 4 can be changed very easily by using the butterfly nuts 26, 26, . . . .

Next, one example of applying a fabric adhesive tape to the affected limb B1 when the external fixation device 1 for fracture treatment is in use will be described.

First, the dog A suffering from a distal radioulnar fracture is given a general anesthetic and hair coat of the entire affected limb B1 is combed carefully. Note that since direct application of a fabric adhesive tape onto the skin causes skin inflammation, the hair coat should not be shaved with a hair clipper so that the hair coat serves as an overcoat.

Then, the ring portion 3 of the external fixation device 1 for fracture treatment is put on periphery C of the shoulder joint by letting the affected limb B1 of the dog A through the ring portion 3. As shown in FIG. 1 and FIG. 5, in this state, as almost the lower half of the ring portion 3 is flexed to the right side (chest side of the dog A), the ring portion 3 is in close contact with the skin of armpit site of the affected limb B1.

Then, an edge D of fracture is reduced as much as possible through flexure and traction.

Then, as shown in FIG. 7(a), to a distal end of radius F and ulna G of the affected limb B1, two front-back fabric adhesive tapes P1, P2 are overlappingly applied down straight from a distal position, about 1 cm away from the edge D of fracture, which is the distal end fracture of radius F and ulna G of the affected limb B1, and are extended in an overlapped state to the lower part of the affected limb B1 to form an extension Q. Two front-back fabric adhesive tapes P3, P4 are overlappingly applied from a proximal position, about 1 cm away from the edge D of fracture to the radial head J in the upper arm direction.

Then, as shown in FIGS. 7(b) and 7(c), reinforcement is provided by horizontally winding two set of upper and lower fabric adhesive tapes P5, P6, and P7, P8 around the distal and proximal ends of the edge D of fracture respectively, ends of the respective fabric adhesive tapes P5 to P8 are pasted.

Then, the affected limb B1 is put loosely into the supporting portion 4 the end R of the extension Q passes from the rear to the front through the long slot 16A of the hook section 16 shown in FIG. 3, and the end R of the extension Q can further pass, from the rear to the front, through the long slot 17A of the locking section 17 and be strongly pulled by the principle of leverage. In this manner, while the affected limb B1 is kept in a strongly pulled state, the extension Q is attached by fabric adhesive tapes and the like and fixed to the locking section 17, thereby keeping the entire affected limb B1 in a fully stretched state, as shown in FIG. 2.

Thus, the traction means 8 provided in the grounding body 7 is not limited to a configuration consisting of the hook section 16 and the locking section 17, and may be such configured that the extension Q can be stretched to keep the affected limb B1 in a pulled state.

Next, as shown in FIG. 7(d), FIG. 1, and FIG. 2, upper and lower fabric adhesive tapes P9, P10 are attached to surround the supporting portion 4 accompanying the affected limb B1 from the radial head J to a distal end of the metacarpal bone H.

In addition, by utilizing the upper arm supporting section 10 which droops from the front of the ring portion 3 to the elbow joint (see point of the elbow K in FIG. 7(d)) of the affected limb B1 and to the front side of the upper arm (see the upper forelimb bone I in FIG. 7(d)), the affected limb B1 is fixed with periphery of the elbow joint pulled forward by upper and lower fabric adhesive tapes P11, P12, so that the elbow joint of the affected limb B1 is not flexed.

Note that as shown in FIG. 3, since a vertically-long slot (vertically-long rectangular opening) 10A is formed in the upper arm supporting section 10, the fabric adhesive tapes P11, P12 can be inversed through the long slot 10A when the affected limb B1 is fixed by the fabric adhesive tapes P11, P12 using the upper arm supporting section 10. Thus, fixation using the fabric adhesive tapes P11, P12 can be performed easily and reliably, and the fixation can be strengthened.

Then, as shown in FIG. 1 and FIG. 2, with the ring portion 3 put on the periphery C of the shoulder joint of the affected limb B1, the ring portion 3 is connected and fixed to the right forelimb B2, which is a forelimb opposite to the affected limb B1, by the fixation means 9.

Specifically, the ring portion 3 is connected and fixed to the right forelimb B2 by the fixing strings 19, 19 by mutually tying one ends of the fixing strings 19, 19, with the fixing strings 19, 19, whose the other ends are locked to the annular strings 18, 18 attached to the ring portion 3, passing through periphery of the armpit of the right forelimb B2.

Note that the fixation means 9 is not limited to the annular strings 18, 18 and the fixing strings 19, 19, and may be such configured by belts, and the like.

Thus, in FIG. 1 showing a state in which the external fixation device 1 for fracture treatment attached to the affected limb B1, since the fracture site (front side of the edge D of fracture) is exposed, palpation as well as X-raying of the fracture site is easy to perform.

With the configuration of the external fixation device 1 for fracture treatment of animals as described above, since with the fracture site of the affected limb B1 loosely put in the supporting portion 4 having the right/left walls 14A, 14B and the back wall 14C and having the upper/lower surfaces and the front face opened, the affected limb B1 is supported by the supporting portion 4 accompanying the affected limb B1 from the radial head to the distal end of the metacarpal bone H, not only the part below the radial head J is stably fixed, but also the fracture site finely and flexibly vibrates and the blood circulation is preserved without being compressed, which thereby can facilitate formation of callus.

Note that since long-term use of the external fixation device 1 for fracture treatment under no weight bearing reduces bone density, thus weakening the bone, the external fixation device 1 for fracture treatment is suitable for fracture treatment which is used for a certain period of time to an extent that the bone density is not reduced, and facilitates formation of callus.

In addition, since the grounding body 7 is provided with the traction means 8 for linear traction of the affected limb B1 downward straight while keeping the entire affected limb B1 stretched, the affected limb B1 is pulled down straight in a stable and reliable manner, thus making it possible to maintain a state in which the edge D of fracture has been reduced, but non-precisely reduced.

In addition, since not only the connecting body 6 connects the rear of the ring portion 3 and the back wall 14C of the supporting portion 4 so that they are firmly integrated, but also the front face of the supporting portion 4 is an opening with no obstruction such as a frame body in front, a veterinarian can easily perform a physical examination, procedure, an X-ray examination and the like, and combined use with the external skeletal fixation method is also allowed.

Furthermore, since the ring portion 3 put on the periphery C of the shoulder joint of the affected limb B1 is connected and fixed to the forelimb B2 opposite to the affected limb B1 by the fixation means 9, the fixed ring portion 3 can be prevented from becoming loose even if an animal (for example, the dog A) loses weight during fracture treatment.

Also, the upper arm supporting section 10 drooping to the elbow joint of the affected limb B1 and to the front side of the upper arm is used, and the elbow joint (see the point of elbow K in FIG. 7(d)) of the affected limb B1 and the upper arm part (see the upper forelimb bone I in FIG. 7(d)) can be fixed to the upper arm supporting section 10 by the fabric adhesive tapes. Thus, the affected limb B1 can be stably supported because the affected limb B1 can be easily pulled down straight with the elbow joint and the upper arm periphery easily fixed.

### Embodiment 2

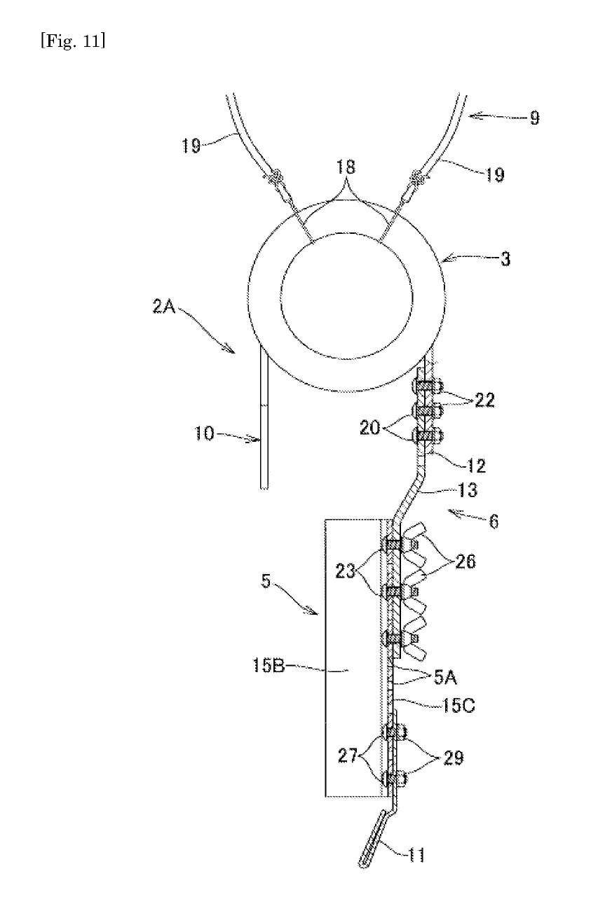

As shown in FIG. 8 to FIG. 13, an external fixation device 2A for fracture treatment of animals according to an embodiment 2 of the present invention is attached to an affected limb B1, for example, a left forelimb, which is an affected limb B1 suffering from a distal radioulnar fracture, of a dog A, and flexibly fixes the affected limb B1, the device including an almost toric ring portion 3 which is to put on periphery C of a shoulder joint and through which the affected limb B1 passes; a supporting portion 5 positioned below the ring portion 3, having right/left walls 15A, 15B and a back wall 15C, having upper/lower surfaces and a front face opened, and accompanying the affected limb B1 from a radial head to a proximal end of metacarpal bone, and supporting the effected limb B1 so that flexible micromotion of fracture site of the affected limb B1 can be produced with it loosely placed in the supporting portion; a connecting body 6 for connecting the rear of the ring portion 3 and the back wall 15C of the supporting portion 5; and a metacarpal pad supporting plate 11 being connected to the back wall 15C of the supporting portion 5, and tilting forward as it accompanies a metacarpal pad L of the affected limb B1 downward from the lower end of the supporting portion 5. The external fixation device 1 for fracture treatment of animals according to the embodiment 1 of the present invention is removed and replaced with the external fixation device 2A for fracture treatment of animals, after callus formation is facilitated by use of the external fixation device 1 for fracture treatment of animals to the extent that bone density is not reduced. Note that the supporting portion 5 has only to accompany the affected limb B1 from the radial head to at least the distal end of the radius. If an upper end of the supporting portion 5 is located above the radial head, a point of the elbow rubs against an inner surface of the back wall 15C of the supporting portion 5. If a lower end of the supporting portion 5 is located above the distal end of radius, fixing of a region below the radial head easily becomes unstable.

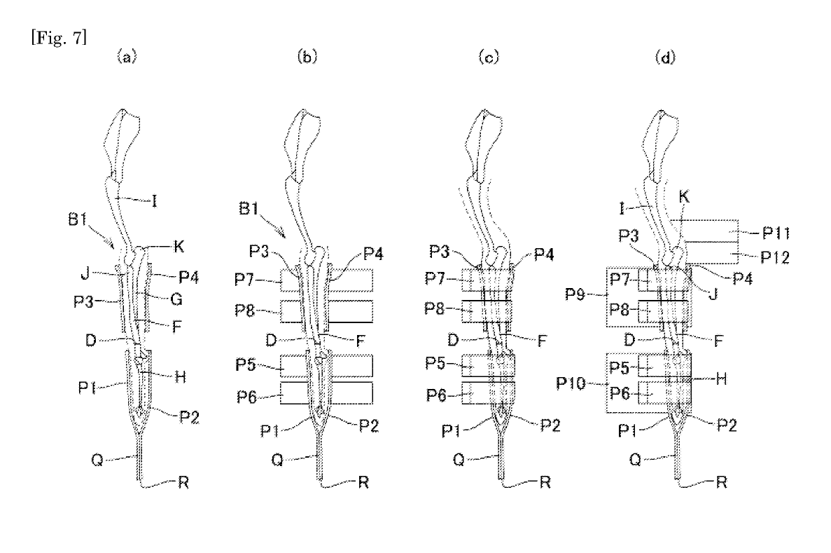

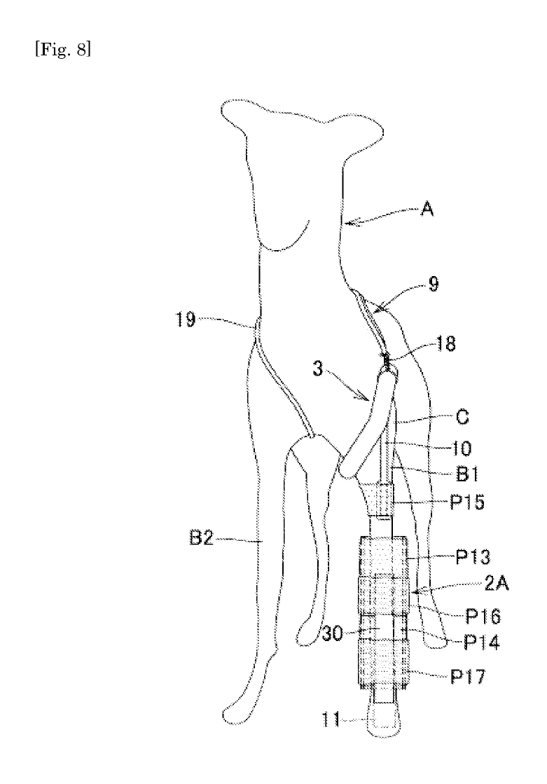

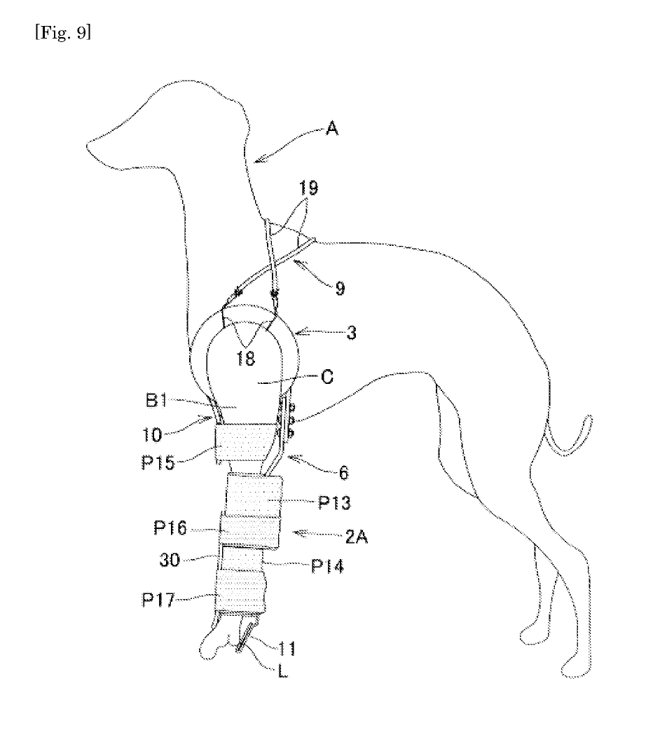

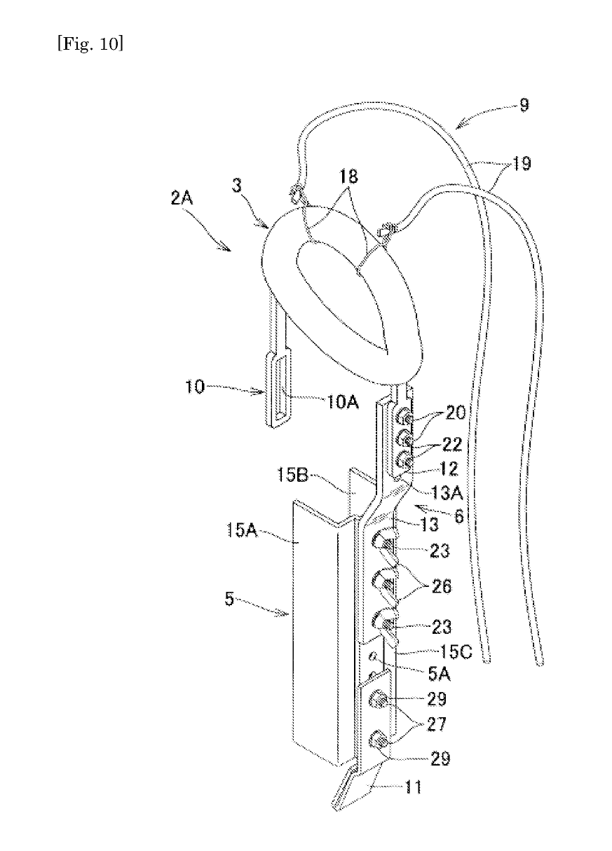

Then, with reference to FIG. 10 to FIG. 13, a structure of the external fixation device 2A for fracture treatment will be described in detail. As same symbols as those in FIG. 3 to FIG. 6 of the embodiment 1 refer to same or corresponding parts, descriptions of components with same symbols as the embodiment 1 will be omitted.

Compared with the external fixation device 1 for fracture treatment of the embodiment 1, the external fixation device 2A for fracture treatment of the embodiment 2 has the supporting portion whose shape is different, has no grounding body 7, and includes a metacarpal pad supporting plate 11.

The supporting portion 5 has a cross section almost shaped like a staple with the front face being open and consisting of a left wall 15A, a right wall 15B, and a back wall 15C. The supporting portion 4 is formed to be long to accompany the affected limb B1 from a radial head to a proximal end of metacarpal bone. As shown in FIG. 13, front-back through-holes 5A, 5A, . . . vertically line up at the center of the right-left direction of the back wall 15C.

In addition, the supporting portion 5 is made of synthetic resin. In particular, the supporting portion 5 made of transparent synthetic resin, such as vinyl chloride resin, polycarbonate or acrylic resin, enables inflammation, swelling, congestion, and the like of the affected limb to be checked. Therefore, it is more preferable embodiment to make the supporting portion of transparent synthetic resin.

The metacarpal pad supporting plate 11 is an aluminum alloy plate, for example. Since front-back through-holes 11A, 11A, . . . vertically line up on the vertically extending plate section, the supporting portion 5 and the metacarpal pad supporting plate 11 are connected and fixed so that they are firmly integrated, by inserting set screws 27 from the front through the through-holes 5A, through-holes 11A, and plain washers 28 to threadably mount the set screws 27 to nuts 29, with the front face of the vertically-extending plate section of the metacarpal pad supporting plate 11 abutting against the lower rear face of the back wall 15C of the supporting portion 5.

Next, one example of cutting a fabric adhesive tape and applying a new fabric adhesive tape when the external fixation device 1 for fracture treatment is removed and replaced with the external fixation device 2A for fracture treatment will be described.

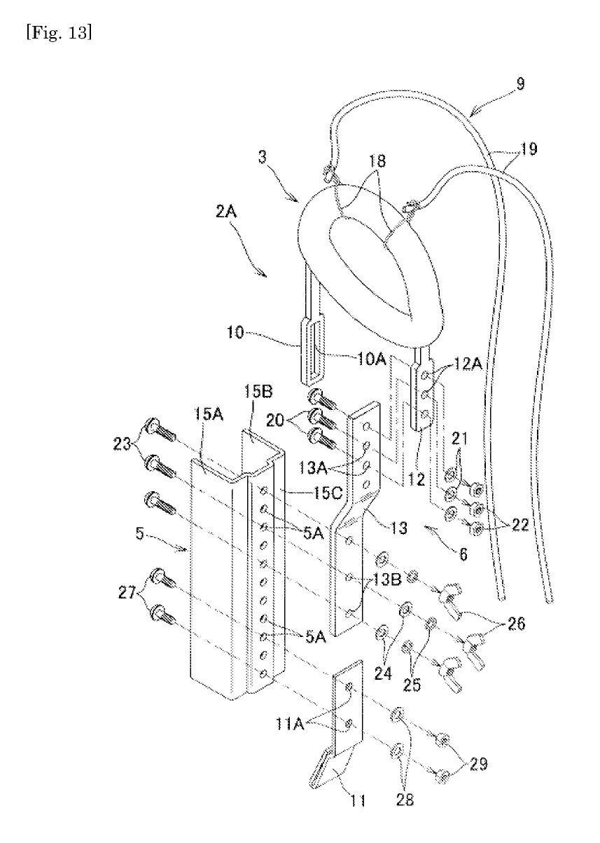

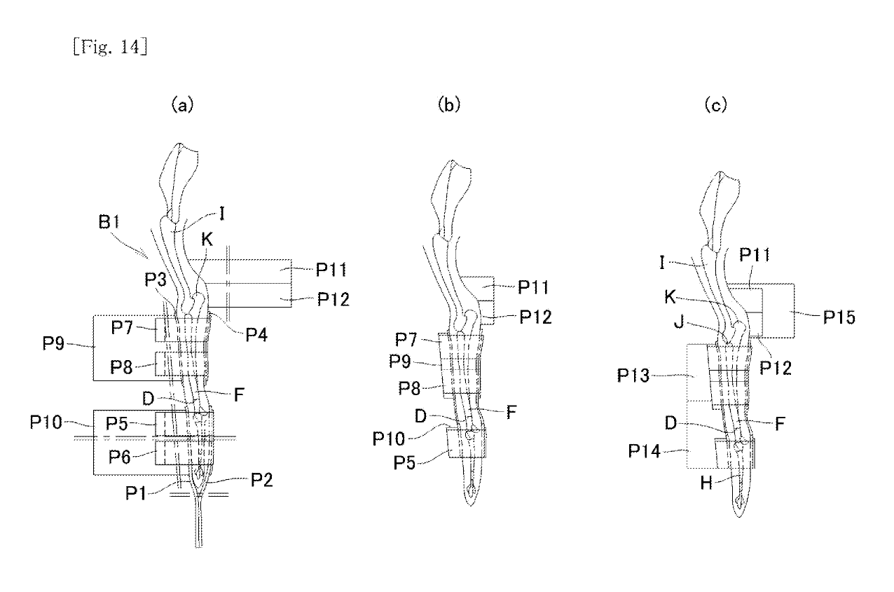

After the external fixation device 1 for fracture treatment is removed from the affected limb B1, as shown in FIG. 14(a), the fabric adhesive tapes P9 to P12 are cut to be in a state shown in FIG. 14(b).

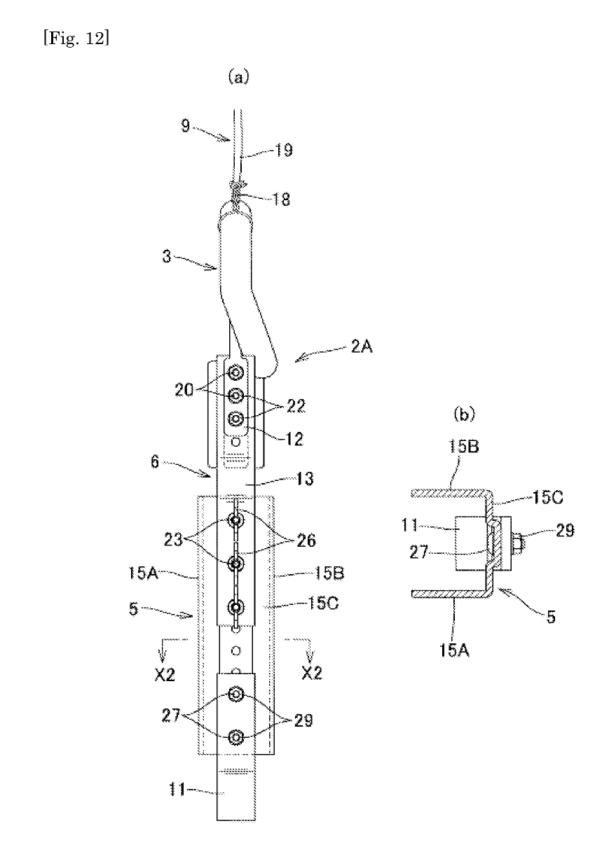

In this state, the ring portion 3 of the external fixation device 2A for fracture treatment is put on the periphery C of the shoulder joint by letting the affected limb B1 of the dog A through the ring portion 3. Then, as shown in FIG. 8 and FIG. 12, as almost the lower half of the ring portion 3 is flexed to the right side (chest side of the dog A), the ring portion 3 is in close contact with the skin of armpit site of the affected limb B1. The affected limb B1 is put loosely into the supporting portion 5, and as shown in FIG. 14(c), FIG. 8, and FIG. 9, the upper and lower fabric adhesive tapes P13, P14 are attached to surround the supporting portion 5 which accompanies the affected limb B1 from the radial head J to the proximal end of the metacarpal bone H.

Then, as shown in FIG. 8 and FIG. 9, reinforcement is provided by attaching to the front face of radius and fixing a splint 30 made of an aluminum alloy plate, for example, whose length is almost equal to length from the radius to carpal joint, by means of fabric adhesive tapes P16, P17 so as to prevent refracture.

In addition, by utilizing the upper arm supporting section 10 which droops from the front of the ring portion 3 to the elbow joint (see point of the elbow K in FIG. 14(c)) of the affected limb B1 and to the front side of the upper arm (see the upper forelimb bone I in FIG. 14(c)), the affected limb B1 is fixed with periphery of the elbow joint pulled forward by the fabric adhesive tape P15, so that the elbow joint of the affected limb B1 is not flexed.

With the configuration of the external fixation device 2A for fracture treatment of animals as described above, since with the fracture site of the affected limb B1 loosely put in the supporting portion 5 having the right/left walls 15A, 15B and the back wall 15C and having the upper/lower surfaces and the front face opened, the affected limb B1 is supported by the supporting portion 5 accompanying from the radial head to the proximal end of the metacarpal bone H, not only the part below the radial head J is stably fixed, but also the fracture site finely vibrates and the blood circulation is preserved without being compressed, which thereby can facilitate formation of callus, and digital pads ground with the metacarpal pad L of the affected limb B1 being supported by the metacarpal pad supporting plate 11. Thus, the device is suitable for fracture treatment in which relatively small weight load is applied and stimulation caused by fine vibration resulting from the weight bearing is provided to the fracture site, which thereby contributes to maturing and increasing of the callus.

### Embodiment 3

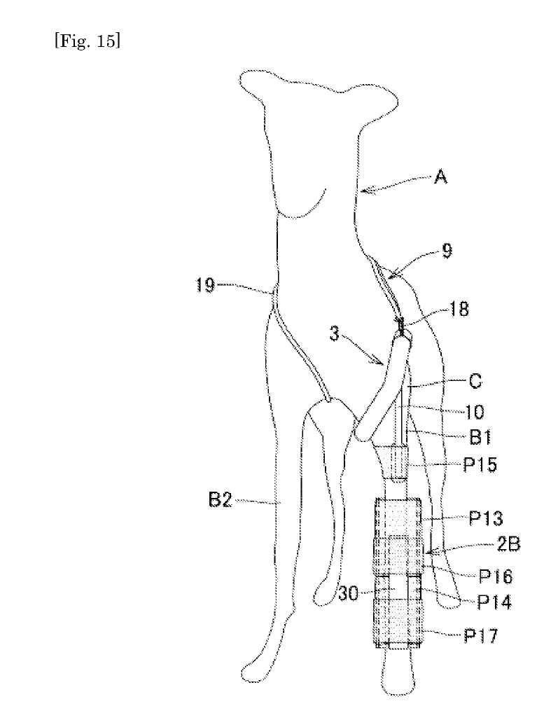

As shown in FIG. 15 and FIG. 16, an external fixation device 2B for fracture treatment of animals according to an embodiment 3 of the present invention is the device without the metacarpal pad supporting plate 11 of the external fixation device 2A for fracture treatment of animals of the embodiment 2 as shown in FIG. 8 to FIG. 13. Callus is matured and increased by using the external fixation device 2A for fracture treatment, and grounding the digital pads, with the metacarpal pad L of the affected limb B1 supported by the metacarpal pad supporting plate 11, while providing stimulation caused by relatively small weight bearing. After a certain amount of callus is formed as the maturing and increasing of the callus advance by effective fracture treatment performed at this stage, the external fixation device 2A for fracture treatment is removed and replaced with the external fixation device 2B for fracture treatment. Alternatively, after the external fixation device 1 for fracture treatment of animals according to the embodiment 1 of the present invention is used to facilitate formation of callus to an extent that bone density is not reduced, the external fixation device 1 for fracture treatment of animals is removed and replaced with the external fixation device 2B for fracture treatment.

While the external fixation device 2B for fracture treatment is the device without the metacarpal pad supporting plate 11 as shown in FIG. 15, FIG. 16, and FIG. 17(b), the metacarpal pad supporting plate 11 bent backward as shown in FIG. 17(c) may be used as the external fixation device 2B for fracture treatment, if the external fixation device 2B for fracture treatment is used after the external fixation device 2A for fracture treatment as shown in FIG. 8, FIG. 9, and FIG. 17(a) is used.

With such a configuration of the external fixation device 2B for fracture treatment of animals, since with a fracture site of the affected limb B1 loosely put in the supporting portion 5 having right/left walls 15A, 15B and a back wall 15C and having upper/lower surfaces and a front face opened, the affected limb B1 is supported by the supporting portion 5 accompanying the affected limb from the radial head J thereof to to proximal end of metacarpal bone H, not only the part below the radial head J is stably fixed, but also the fracture site finely vibrates and the blood circulation is preserved without being compressed, which thereby can facilitate formation of callus, and a metacarpal pad L and digital pads of the affected limb B1 ground. Thus, the device is suitable for fracture treatment in which large weight load is applied, and stimulation caused by fine vibration resulting from the weight bearing is provided to the fracture site, which thereby contributes to maturing and increasing the callus.

While the above description shows the case in which the cross section of the supporting portion 4 consisting of the left wall 14A, the right wall 14B, and the back wall 14C and the cross section of the supporting portion 5 consisting of the left wall 15A, the right wall 15B, and the back wall 15C are almost shaped like a staple, the supporting portions 4 and 5 are not limited to those having such a cross sectional shape, and the cross section may be almost shaped like a letter U or letter C (almost gutter-shaped) having the front face opened, and the like.

Next, a method for treating fractures of animals using the external fixation devices 1, 2A, 2B for fracture treatment of animals according to the embodiments of the present invention will be described.

### (First Fracture Treatment Method)

A first fracture treatment method of the present invention includes:

(1) A reduction step of reducing an affected limb B1 by linear traction or toggling an edge of fracture;
(2) a callus formation facilitation step of facilitating formation of callus to an extent that bone density is not reduced, with the use of the external fixation device 1 for fracture treatment of animals of the embodiment 1, by linear traction of the affected limb B1 by traction means 8 and grounding the grounding body 7;
(3) a callus maturing and increasing step based on weight bearing of maturing and increasing callus, with the use of the external fixation device 2A for fracture treatment of animals of the embodiment 2, by grounding digital pads of the affected limb B1 to provide a fracture site with stimulation resulting from the weight bearing; and
(4) a callus maturing and increasing step based on weight bearing of maturing and increasing callus, with the use of the external fixation device 2B for fracture treatment of animals of the embodiment 3, by grounding a metacarpal pad L and the digital pads of the affected limb B1 to provide the fracture site with stimulation resulting from the weight bearing.

Note that the reduction step (1) is performed with a mild degree of a general anesthetic or under sedation. Reduction may also be possible with no use of an anesthetic when a quadruped such as a dog A is docile. If external skeletal fixation is used in combination, the external skeletal fixation is performed in this step with use of a general anesthetic.

In addition, application of fabric adhesive tapes to the affected limb B1 or attachment of the external fixation devices 1, 2A, 2B for fracture treatment is also performed with a mild degree of a general anesthetic or under sedation. It may also be possible with no use of an anesthetic when a quadruped such as a dog A is docile.

With such a fracture treatment method, after the callus formation is facilitated by using the external fixation device 1 for fracture treatment to the extent that bone density is not reduced, the external fixation device 1 for fracture treatment is removed and replaced with the external fixation device 2A for fracture treatment, thereby grounding the digital pads with the metacarpal pad L of the affected limb B1 being supported by the metacarpal pad supporting plate 11 to mature and increase the callus while providing stimulation caused by fine vibration resulting from relatively small weight bearing. Effective fracture treatment is performed at this stage, so that maturing and increasing of the callus advance to a state in which a certain amount of the callus is formed. Then, the external fixation device 2A for fracture treatment is removed and replaced with the external fixation device 2B for fracture treatment, thereby grounding the metacarpal pad L and the digital pads of the affected limb B1 to mature and increase the callus while providing stimulation caused by fine vibration resulting from relatively large weight bearing.

The fracture treatment method to be performed by using in a phased manner the external fixation device 1 for fracture treatment, the external fixation device 2A for fracture treatment, and the external fixation device 2B for fracture treatment not only allows safe and early healing involving full recovery of functions but also enables the risk of refracture to be minimized.

### (Second Fracture Treatment Method)

A second fracture treatment method of the present invention includes:

(1) A reduction step of reducing an affected limb B1 by linear traction or toggling an edge of fracture;
(2) a callus formation facilitation step of facilitating formation of callus to an extent that bone density is not reduced, with the use of the external fixation device 1 for fracture treatment of animals of the embodiment 1, by linear traction of the affected limb B1 by traction means 8 and grounding the grounding body 7; and
(3) a callus maturing and increasing step based on weight bearing of maturing and increasing callus, with the use of the external fixation device 2B for fracture treatment of animals of the embodiment 3, by grounding a metacarpal pad L and digital pads of the affected limb B1 to provide the fracture site with stimulation resulting from the weight bearing.

Note that the reduction step (1) is performed with a mild degree of a general anesthetic or under sedation. Reduction may also be possible with no use of an anesthetic when a quadruped such as a dog A is docile. If external skeletal fixation is used in combination, the external skeletal fixation is performed in this step with use of a general anesthetic.

In addition, application of fabric adhesive tapes to the affected limb B1 or attachment of the external fixation devices 1, 2A, 2B for fracture treatment is also performed with a mild degree of a general anesthetic or under sedation. It may also be possible with no use of an anesthetic when a quadruped such as a dog A is docile.

With such a fracture treatment method, if a certain amount of callus has already been formed as the formation of callus has already facilitated by use of the external fixation device 1 for fracture treatment to the extent that the bone density is not reduced, that is to say, when formation of callus is fast in a fracture of a young dog, for example, the external fixation device 1 for fracture treatment is removed and replaced with the external fixation device 2B for fracture treatment, grounding the metacarpal pad L and the digital pads of the affected limb B1, and providing stimulation caused by fine vibration resulting from relatively large weight bearing to mature and increase callus, thereby being able to perform effective fracture treatment.

The fracture treatment method to be performed by using in a phased manner the external fixation device 1 for fracture treatment and the external fixation device 2B for fracture treatment not only allows safe and early healing involving full recovery of functions but also enables the risk of refracture to be minimized.

In addition, with the first fracture treatment method or the second fracture treatment method as described above, the connecting body 6 connects the rear of the ring portion 3 and the back walls 14C,15C of the supporting portions 4, 5 so that they are firmly integrated, and the front faces of the supporting portions 4, 5 are an opening with no obstruction such as a frame body in front. Thus, a veterinarian can easily perform a physical examination, procedure, an X-ray examination and the like, and the external fixation devices 1, 2A, 2B for fracture treatment to be used in the fracture treatment methods can be used in combination with the external skeletal fixation method.

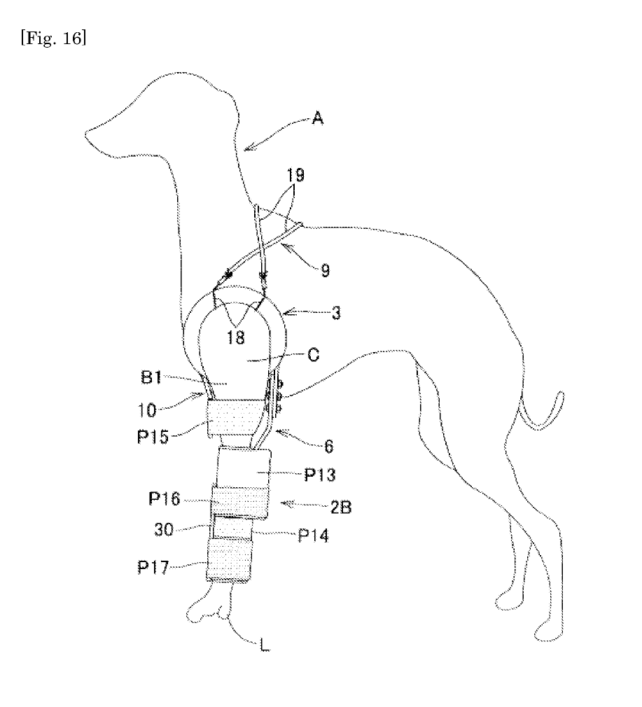

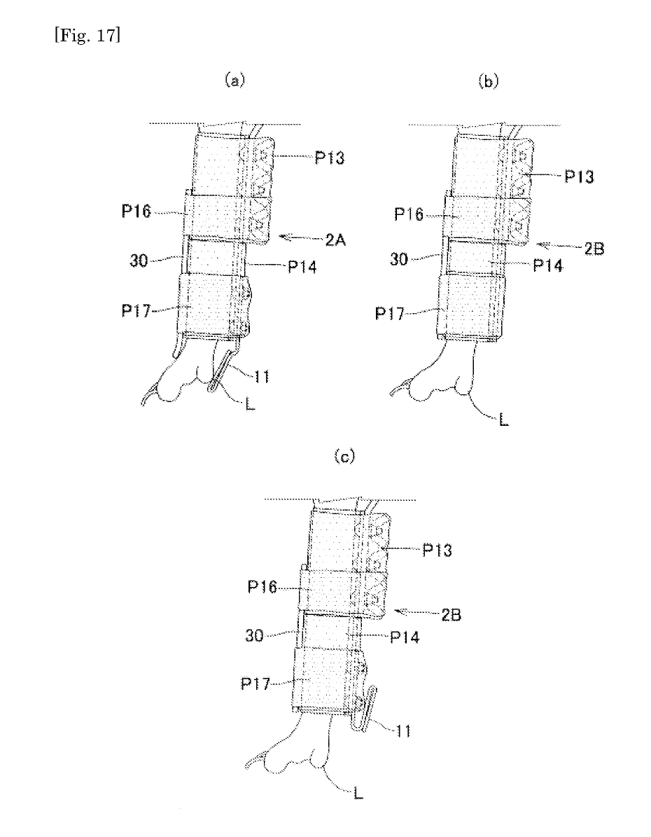

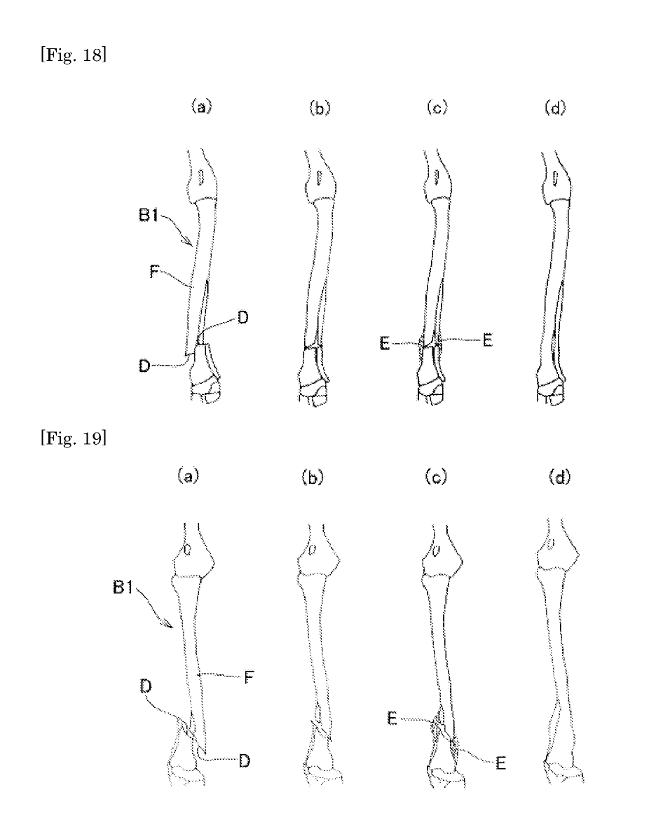

As shown in the schematic views of X-ray images in FIG. 18 and FIG. 19, when a distal end of radius F is fractured (edge D of fracture) as in FIG. 18(a) and FIG. 19(a), and the reduction step (1) of the first fracture treatment method or the second fracture treatment method of the present invention is performed, the radius F comes to the state shown in FIG. 18(b) and FIG. 19(b). If the callus formation facilitation step (2) and the callus maturing and increasing steps (3) and (4) based on the weight bearing of the first fracture treatment method of the present invention, or the callus formation facilitation step (2) and the callus maturing and increasing step (3) based on the weight bearing of the second fracture treatment method of the present invention are performed, a bridge-like calluses E as shown in FIG. 18(c) and FIG. 19(c) are formed, and then, the bone is remodeled subsequently, as shown in FIG. 18(d) and FIG. 19(d).

### Embodiment 1

### Target of Treatment

(1) Dog type: Toy Poodle

### (2) Sex: Male

### (3) Weight: 3.0 kg

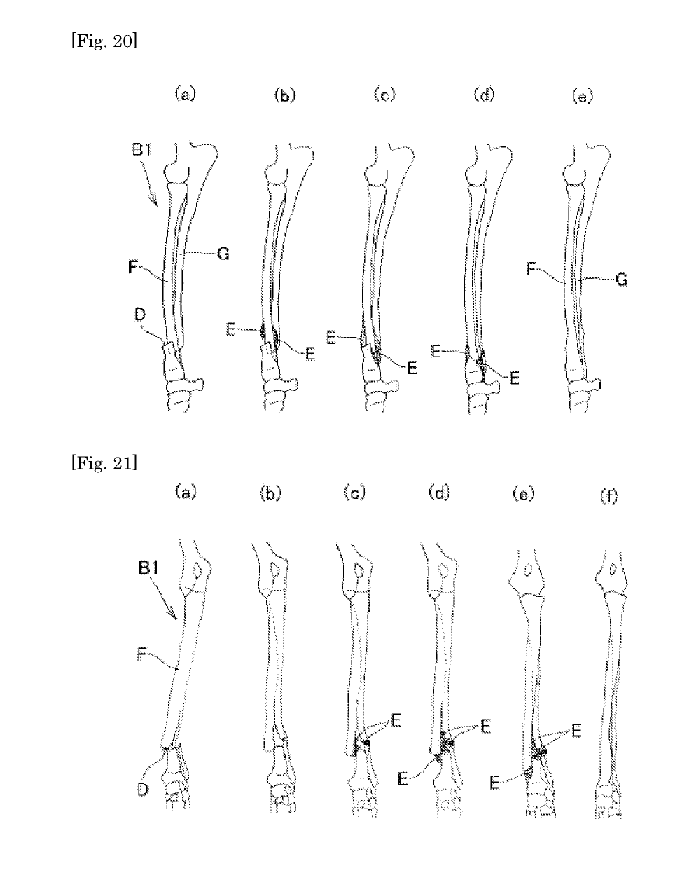

(4) Age in month at the time of injury: 10 months
(5) Type of fracture: Transverse fracture of left radius and ulna due to flexural load at a site of approximately 18% from a distal end of the radius (See the edge D of fracture in FIG. 20(a) and FIG. 21(a).)

### (Treatment Method and Progress)

First, after reduction was performed as much as possible, as shown in FIG. 21(b), treatment was provided by using the external fixation device 1 for fracture treatment. Through the state shown in FIG. 20(b) and the state shown in FIG. 21(c), on the 28^{th} day of illness, calluses E as shown in FIG. 20(c) and FIG. 21(d) were formed.

Then, treatment was provided by removing the external fixation device 1 for fracture treatment and replacing it with the external fixation device 2B for fracture treatment. On the 42^{nd} day of illness, as sufficient calluses E as shown in FIG. 20(d) and FIG. 21(e) were formed, the external fixation device 2B for fracture treatment was removed and the fracture was healed.

After six months, as shown in FIG. 20(e) and FIG. 21(f), no malunion was observed between radius F and ulna G, and the bones were remodeled so that the radius F and the ulna G were separate.

### Embodiment 2

### Target of Treatment

(1) Dog type: Toy Poodle

### (2) Sex: Male

### (3) Weight: 2.1 kg

(4) Age in month at the time of injury: 10 months
(5) Type of fracture: Oblique fracture of right radius and ulna due to compressive load at a site of approximately 10% from a distal end of the radius (See the edge D of fracture in FIG. 22(a) and FIG. 23(a).)

### (Treatment Method and Progress)

First, after reduction was performed as much as possible, as shown in FIG. 22(b) and FIG. 23(b), treatment was provided by using the external fixation device 1 for fracture treatment. On the 42nd day of illness, calluses E as shown in FIG. 22(c) and FIG. 23(c) were formed.

Then, treatment was provided by removing the external fixation device 1 for fracture treatment and replacing it with the external fixation device 2A for fracture treatment. On the 63^{rd} day of illness, sufficient calluses E as shown in FIG. 22(d) and FIG. 23(d) were formed.

Then, treatment was provided by removing the external fixation device 2A for fracture treatment and replacing it with the external fixation device 2B for fracture treatment. On 91^{st} day of illness, as sufficient calluses E as shown in FIG. 22(e) and FIG. 23(e) were formed, the external fixation device 2B for fracture treatment was removed and the fracture was healed.

After six months, as shown in FIG. 22(f) and FIG. 23(f), no malunion was observed between radius F and ulna G, and the bones were remodeled so that the radius F and the ulna G were separate.

### Embodiment 3

### Target of Treatment

(1) Dog type: Chihuahua

### (2) Sex: Female

### (3) Weight: 2.0 kg

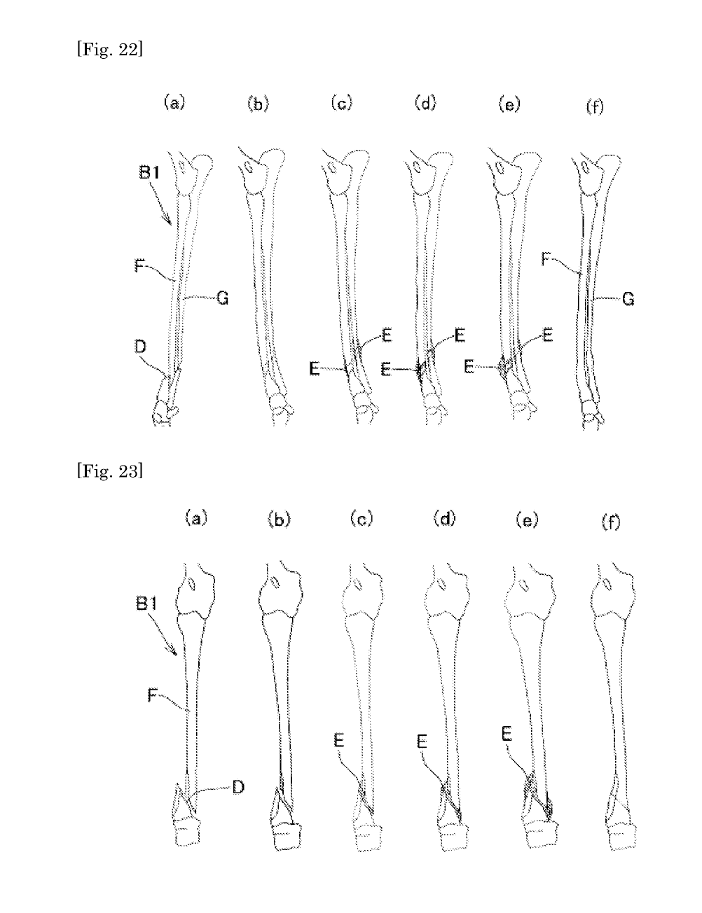

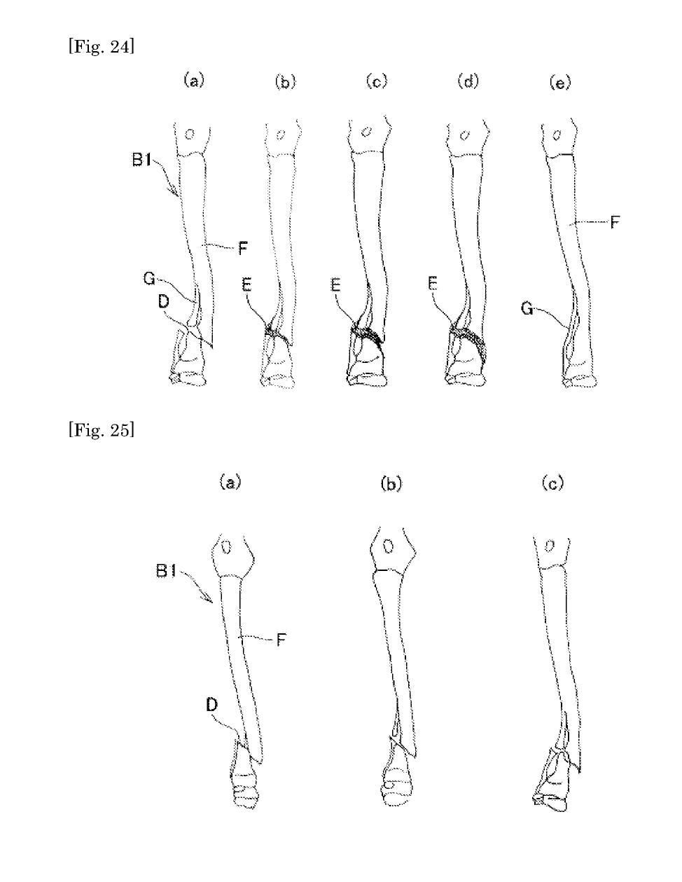

(4) Age in month at the time of injury: 11 months
(5) Type of fracture: Oblique fracture of right radius and ulna due to compressive load at a site of approximately 17% from a distal end of the radius (See the edge D of fracture in FIG. 24(a).)

### (Treatment Method and Progress)

First, in the state in FIG. 24(a), which is nonunion after being subjected to the fracture treatment of the Comparative Example 1 to be described later, treatment was provided by using the external fixation device 1 for fracture treatment. On the 21^{st} day of illness, the calluses E as shown in FIG. 24(b) were formed.

Then, treatment was provided by removing the external fixation device 1 for fracture treatment and replacing it with the external fixation device 2A for fracture treatment. On the 42^{nd} day of illness, the calluses E as shown in FIG. 24(c) were formed.

Then, treatment was provided by removing the external fixation device 2A for fracture treatment and replacing it with the external fixation device 2B for fracture treatment. On the 56^{th} day of illness, as sufficient calluses E as shown in FIG. 24(d) were formed, the external fixation device 2B for fracture treatment was removed and the fracture was healed.

After three months, as shown in FIG. 24(e), no malunion was observed between radius F and ulna G, and the bones were remodeled so that the radius F and the ulna G were separate.

### Comparative Example 1

### Target of Treatment

(1) Dog type: Chihuahua

### (2) Sex: Female

### (3) Weight: 2.0 kg

(4) Age in month at the time of injury: 11 months
(5) Type of fracture: Oblique fracture of right radius and ulna due to compressive load at a site of approximately 17% from a distal end of the radius (See the edge D of fracture in FIG. 25(a).)

### (Treatment Method and Progress)

First, after reduction, treatment was provided by using the conventional type-A external fixation device. On the 35^{th} day of illness, as shown in FIG. 25(b), no formation of callus was observed. Thus, the type-A external fixation device was removed and fixation was performed with only the gutter-like splint (letter C shape of the horizontal cross section) made of synthetic resin.

On the 42^{nd} day of illness, as shown in FIG. 25(c), no formation of callus was observed and nonunion was found.

### Comparative Example 2

### Target of Treatment

(1) Dog type: Toy Poodle

### (2) Sex: Female

### (3) Weight: 3.0 kg

(4) Age in month at the time of injury: 24 months
(5) Type of fracture: Oblique fracture of right radius and ulna at a site of approximately 13% from a distal end of the radius

### (Treatment Method and Progress)

After the reduction, treatment was provided by using the conventional type-A external fixation device. On the 42^{nd} day of illness, no formation of callus was observed and nonunion was found.

### Comparative Example 3

### Target of Treatment

(1) Dog type: Cavalier

### (2) Sex: Male

### (3) Weight: 8.0 kg

(4) Age in month at the time of injury: 12 months
(5) Type of fracture: Comminuted fracture of left radius and ulna at a site of approximately ⅓ from a distal end of the radius

### (Treatment Method and Progress)

Although a surgery was performed with the plate internal fixation method, the screw inserted into the hole of the plate became loose on the 28^{th} day of illness. Simultaneously, the plate also became loose, and nonunion was found.

In the above, the description was provided by taking the dog A for example of a quadruped. However, in addition to cats and dogs, quadrupeds to which the present invention is applied include calf, colt, lamb, and the like, the economic value of which is significantly damaged by forelimb fractures at the time of or after birth.

Specifically, for pet animals such as small dogs or kittens, the present invention is of a high practical value because of a shortened period of fracture treatment, prevention of refracture, reduction of treatment costs and the like. In addition, being applied to industrial animals, since the present invention can heal those that would be otherwise disposed in a shorter period of time and at a moderate price, the economic effect of the present invention is extremely high.

### REFERENCE SIGNS LIST

A Dog (Quadruped)
B1 Affected limb (Left forelimb)
B2 Right forelimb
C Periphery of shoulder joint
D Edge of fracture
E Callus
F Radius
G Ulna
H Metacarpal bone
I Upper forelimb bone
J Radial head
K Point of the elbow
L Metacarpal pad
P1 to P17 Fabric adhesive tape
Q Extension
R End
1, 2A, 2B External fixation device for fracture treatment
3 Ring portion
4,5 Supporting portion
4A, 5A Through-hole
6 Connecting body
7 Grounding body
7A Through-hole
8 Traction means
9 Fixation means
10 Upper arm supporting section
10A Long slot
11 Metacarpal pad supporting plate
11A Through-hole
12 Mounting plate
12A Through-hole
13 Connecting plate
13A, 13B Through-hole
14A Left wall
14B Right wall
14C Back wall
15A Left wall
15B Right wall
15C Back wall
16 Hook section
16A Long slot
17 Locking section
17A Long slot
18 Annular string
19 Fixing string
20 Set screw
21 Plain washer
22 Nut
23 Set screw
24 Plain washer
25 Spring washer
26 Butterfly nut
27 Set screw
28 Plain washer
29 Nut
30 Splint

1-11. (canceled)

12. An external fixation device for fracture treatment used in treatment of forelimb fractures of quadrupeds for fixing an affected limb, comprising:
an almost toric ring portion which is to put on periphery of a shoulder joint and through which the affected limb passes;
a supporting portion having right/left walls and a back wall, having upper/lower surfaces and a front face opened, and accompanying the affected limb from a radial head to at least a distal end of radius, and supporting the affected limb so that flexible micromotion of fracture site of the affected limb can be produced with it loosely placed in the supporting portion;
a connecting body for connecting the rear of the ring portion and the back wall of the supporting portion; and
a grounding body being connected to the back wall of the supporting portion and positioned below the opening of the lower surface of the supporting portion, and including traction means for linear traction of the affected limb downward straight while keeping the entire affected limb stretched.

13. An external fixation device for fracture treatment used in treatment of forelimb fractures of quadrupeds for fixing an affected limb, comprising:
an almost toric ring portion which is to put on periphery of a shoulder joint and through which the affected limb passes;
a supporting portion having right/left walls and a back wall, having upper/lower surfaces and a front face opened, and accompanying the affected limb from a radial head to at least a distal end of radius, and supporting the affected limb so that flexible micromotion of fracture site of the affected limb can be produced with it loosely placed in the supporting portion;
a connecting body for connecting the rear of the ring portion and the back wall of the supporting portion; and
a metacarpal pad supporting plate being connected to the back wall of the supporting portion, and tilting forward as it accompanies a metacarpal pad of the affected limb downward from the lower end of the supporting portion.

14. An external fixation device for fracture treatment used in treatment of forelimb fractures of quadrupeds for fixing an affected limb, comprising:
an almost toric ring portion which is to put on periphery of a shoulder joint and through which the affected limb passes;
a supporting portion having right/left walls and a back wall, having upper/lower surfaces and a front face opened, and accompanying the affected limb from a radial head to at least a distal end of radius, and supporting the affected limb so that flexible micromotion of fracture site of the affected limb can be produced with it loosely placed in the supporting portion; and
a connecting body for connecting the rear of the ring portion and the back wall of the supporting portion.

15. The external fixation device for fracture treatment of animals according to claim 12, comprising fixation means to connect and fix the ring portion to a forelimb opposite to the affected limb, with the ring portion put on the periphery of the shoulder joint of the affected limb.

16. The external fixation device for fracture treatment of animals according to claim 12, comprising an upper arm supporting section drooping from the front of the ring portion to an elbow joint of the affected limb and the front side of the upper arm.

17. The external fixation device for fracture treatment of animals according to claim 12, wherein the supporting portion is made of transparent synthetic resin.

18. A method for treating forelimb fractures of quadrupeds, comprising:
a reduction step of reducing an affected limb by linear traction or toggling an edge of the fracture;
a callus formation facilitation step of facilitating formation of callus to an extent that bone density is not reduced, with the use of the external fixation device for fracture treatment of animals of claim 12, by linear traction of the affected limb with the traction means and grounding the grounding body.

19. A method for treating forelimb fractures of quadrupeds, comprising:
a reduction step of reducing an affected limb by linear traction or toggling an edge of the fracture;
a callus maturing and increasing step based on weight bearing of maturing and increasing callus, with the use of the external fixation device for fracture treatment of animals of claim 13, by grounding digital pads of the affected limb to provide the fracture site with stimulation resulting from the weight bearing.

20. A method for treating forelimb fractures of quadrupeds, comprising:
a reduction step of reducing an affected limb by linear traction or toggling an edge of the fracture;
a callus maturing and increasing step based on weight bearing of maturing and increasing callus, with the use of the external fixation device for fracture treatment of animals of claim 14, by grounding a metacarpal pad and the digital pads of the affected limb to provide the fracture site with stimulation resulting from the weight bearing.

21. A method for treating forelimb fractures of quadrupeds, comprising:
a reduction step of reducing an affected limb by linear traction or toggling an edge of the fracture;
a callus formation facilitation step of facilitating formation of callus to an extent that bone density is not reduced, with the use of the external fixation device for fracture treatment of animals of the claim 12, by linear traction of the affected limb with the traction means and grounding the grounding body.

22. A method for treating forelimb fractures of quadrupeds, comprising:
a reduction step of reducing an affected limb by linear traction or toggling an edge of the fracture;
a callus maturing and increasing step based on weight bearing of maturing and increasing callus, with the use of the external fixation device for fracture treatment of animals of claim 14, by grounding a metacarpal pad and digital pads of the affected limb to provide the fracture site with stimulation resulting from the weight bearing.

23. The method for treating fractures of animals according to claim 18, comprising fixation means to connect and fix the ring portion to a forelimb opposite to the affected limb, with the ring portion of the external fixation device for fracture treatment put on the periphery of the shoulder joint of the affected limb.

24. The method for treating fractures of animals according to claim 18, comprising an upper arm supporting section drooping from the front of the ring portion of the external fixation device for fracture treatment to the elbow joint of the affected limb and the front side of the upper arm.

25. The method for treating fractures of animals, according to claim 18, wherein the supporting portion of the external fixation device for fracture treatment is made of transparent synthetic resin.
